(19) United States
(12) Patent Application Publication
(10) Pub. No.: US 2026/0192033 A1
(43) Pub. Date: Jul. 9, 2026

(54) Medical Waste Collection System For Collecting Medical Waste During A Medical Procedure

(71) Applicant: Stryker Corporation, Portage, MI (US)

(72) Inventors: Steven J. Kaplan, Kalamazoo, MI (US); Joshua Caleb Colvin, Paw Paw, MI (US); Fernando Erismann, Sacramento, CA (US); Clayton H. Meyers, Paw Paw, MI (US); Michael Zollinger, Chelsea, MI (US); Grant Westphal, Delton, MI (US)

(73) Assignee: Stryker Corporation, Portage, MI (US)

(21) Appl. No.: 19/129,360

(22) PCT Filed: Nov. 13, 2023
(86) PCT No.: PCT/US2023/037188

§ 371 Date: May 13, 2025

Related U.S. Application Data
(60) Provisional application No. 63/425,098, filed on Nov. 14, 2022.
(60) Provisional application No. 63/454,860, filed on Mar. 27, 2023.

(51) Int. Cl. A61M 1/00

(52) U.S. Cl. CPC A61M 1/60; A61M 1/73; A61M 2205/3389; A61M 2205/52; A61M 2205/587

(57) ABSTRACT

A medical waste collection system for collecting waste material. A level sensor is coupled to at least one waste container to generate a signal indicating a fluid level of waste material therein. A controller is configured to operate a light source assembly based on the signal such that a portion of the interior of the waste container disposed beneath the fluid level of the waste material is illuminated. A cover assembly may include a rack and pinion and/or polarizing segments to selectively limit visibility of portions of the waste container. The controller may initiate an unclogging protocol to create a pressure differential between an upper waste container and a lower waste container based on a determination that a transfer valve between the waste containers may be clogged. The determination may be based on a rate of change of the fluid level as measured by the level sensor(s).

### CROSS REFERENCE TO RELATED APPLICATIONS

This application claims priority to and all the benefits of U.S. Provisional Application No. 63/454,860, filed Mar. 27, 2023, and U.S. Provisional Application No. 63/425,098, filed Nov. 14, 2022, the entire contents of each being hereby incorporated by reference.

### BACKGROUND

Medical waste collection systems are used in health care facilities to collect waste material generated during a medical procedure. A medical waste collection system may include one or more waste containers in fluid communication with a vacuum source. When the vacuum source is operating, waste material is drawn through suction lines and into the waste container(s).

The waste container may be viewable through windows, and the waste container may be at least partially formed from a clear material. In some instances, a user may wish to view the waste material within the waste container. A light source may be provided to illuminate the waste container. However, such light sources often illuminate portions of the waste container that are undesirable to view, for example, a soiled sidewall of the clear material. In other instances, the user may wish to obscure or hide the waste container from view, and a cover may be employed to selectively shield the waste containers from view. In such situations, it can be difficult to accommodate an actuation assembly and the cover in a system that is designed to be compact.

One exemplary medical waste collection system is sold under the tradename Neptune by Stryker Corporation (Kalamazoo, Mich.), in which an upper waste container is disposed above a lower waste container. The upper waste container has a volume of four liters, whereas the lower waste container has a volume of twenty liters. While independent suction may be simultaneously drawn on the upper and lower waste containers, often in operation, the upper waste container is primarily used. The Neptune system provides for transferring or emptying the upper waste container into the lower waste container through a transfer valve, a process also known as “dumping.” In some cases, the waste material may partially or fully clog the path or volume near or within the transfer valve. For example, a clog may result from blood clots and other buildup of waste material within or near the transfer valve. This could lead to delay in resuming the medical procedure, and/or downtime of the medical waste collection system for service.

Therefore, there is a need in the art for a medical waste collection system capable of overcoming one or more of the aforementioned shortcomings.

### SUMMARY

A first aspect includes a medical waste collection system for collecting waste material. The medical waste collection system includes a waste container. The system also includes a vacuum source configured to provide a vacuum on the waste container. The system also includes a chassis supporting the waste container and the vacuum source. The system also includes a level sensor coupled to the waste container and configured to generate a signal indicative of a fluid level of the waste material disposed within the waste container. The system also includes a light source assembly that may include a plurality of light sources, each of the plurality of light sources may be configured to be selectively operable to illuminate an interior of the waste container. The system also includes a controller coupled to the level sensor and the light source assembly. The controller may be configured to operate the plurality of light sources based on the signal such that a portion of the interior of the waste container disposed beneath the fluid level of the waste material is illuminated.

A second aspect includes a medical waste collection system for collecting waste material. The medical waste collection system includes a waste container. The system also includes a vacuum source configured to provide a vacuum on the waste container. The system also includes a chassis supporting the waste container and the vacuum source. The system also includes an ambient light sensor configured to generate a signal indicative of an intensity of ambient light. The system also includes a light source positioned emit light of variable intensity into the waste container. The system also includes a controller coupled to the sensor and the light source. The controller is configured to operate the light source to illuminate an interior of the waste container at a determined intensity based on the signal received from the ambient light sensor.

A third aspect includes a medical waste collection system for collecting waste material. The medical waste collection system includes a first waste container defining a first interior for collecting the waste material. The system also includes a second waste container defining a second interior for collecting the waste material. The system also includes a transfer valve coupled to the first and second waste containers and configured to permit waste material to be transferred to the second waste container from the first waste container. The system also includes a vacuum source configured to provide a vacuum on at least one of the first waste container and the second waste container. The system also includes a chassis supporting the first and second waste containers, the vacuum source, and the transfer valve. The system also includes a level sensor coupled to at least one of the first waste container and the second waste container and configured to generate a signal indicative of a fluid level of the waste material disposed within a respective one of the first and second waste containers. The system also includes a light source configured to be selectively operable to illuminate the interior of a respective one of the first and second waste containers. The system also includes a controller coupled to the transfer valve, the level sensor, and the light source. The controller is configured to operate the transfer valve to initiate a fluid transfer, receive subsequent signals from the level sensor indicative of a corresponding change in the fluid level in at least one of the first and second waste containers, and operate the light source to indicate a clogged condition in which a rate of increase of the fluid level in the second waste container is below a preset fluid transfer rate.

A fourth aspect includes a medical waste collection system for collecting waste material. The medical waste collection system includes a waste container. The system also includes a vacuum source configured to provide a vacuum on the waste container at a selected vacuum level. The system also includes a chassis supporting the waste container and the vacuum source. The system also includes a level sensor coupled to the waste container and configured to generate fluid level signals indicative of a fluid level of the waste material disposed within the waste container. The system also includes a light source positioned to emit light of variable intensity into the waste container. The system also includes a controller coupled to the sensor and the light source. The controller is configured to receive vacuum signals indicative of the vacuum level, receive the fluid level signals, and operate the light source to indicate an insufficient volume condition in which an anticipated rate of increase of the fluid level is expected to consume an available volume of the waste container in less than a predetermined period.

A fifth aspect includes a method of operating a medical waste collection system in communication with a quantitative blood loss (QBL) system. The method includes operating the vacuum source to draw medical waste into the waste container. The method also includes receiving, at the controller, QBL data from the QBL system, where the QBL data is indicative of a blood concentration within the medical waste. The method also includes receiving, at the controller, fluid level signals from the level sensor. The method also includes determining, with the controller, a fluid volume of the medical waste within the waste container. The method also includes determining, with the controller, a lost blood volume based on the blood concentration and the fluid volume. The method also includes operating the light source to indicate a warning condition in which the lost blood volume deviates beyond a predetermined threshold.

A sixth aspect includes a medical waste collection system for collecting waste material. The medical waste collection system includes a waste container may include a transparent material. The system also includes a vacuum source configured to provide a vacuum on the waste container. The system also includes a chassis supporting the waste container and the vacuum source. The chassis may include a housing defining a viewing window through which the waste container is viewable. The system also includes a cover assembly coupled to the housing and may include a first cover may include a first polarizer, and a second cover moveable relative to the first cover. The cover assembly also includes a second polarizer having a different polarizing orientation than the first polarizer, and an actuator coupled to the first cover or the second cover and configured to receive an input to move the first cover and the second cover relative to one another to selectively reduce light being transmitted through the cover assembly based on misalignment of the first and second polarizers.

A seventh aspect includes a medical waste collection system for collecting waste material. The medical waste collection system includes a waste container. The system also includes a vacuum source configured to provide a vacuum on the waste container. The system also includes a chassis for supporting the waste container and the vacuum source. The chassis may include a housing defining a viewing window through which the waste container is viewable. The system also includes a cover assembly coupled to the housing and may include a first cover having a first rack, a second cover having a second rack, and a pinion disposed between the first and second covers and engaged with the first and second racks such that movement of first cover in one direction causes movement of the second cover in an opposite direction. The system also includes an actuator coupled to one of the first cover and the pinion and configured to receive an input to move the first and second covers towards one another to selectively occlude at least a portion of the window.

An eighth aspect includes a medical waste collection system for collecting waste material. The medical waste collection system includes a waste container. The system also includes a vacuum source configured to provide a vacuum on the waste container. The system also includes a chassis for supporting the waste container and the vacuum source. The chassis may include a housing defining a viewing window through which the waste container is viewable. The system also includes a cover assembly coupled to the housing and may include a flexible cover movably coupled to one of the housing and the chassis, first and second support rods rotatably coupled to one of the housing and the chassis, and a belt coupled to the cover and configured to surround the support rods. The system also includes an actuator coupled to one of the belt and the cover and configured to receive an input to move the cover to selectively occlude at least a portion of the window.

A ninth aspect includes a medical waste collection system for collecting waste material. The medical waste collection system includes a waste container. The system also includes a vacuum source configured to provide a vacuum on the waste container. The system also includes a chassis for supporting the waste container and the vacuum source. The chassis may include a housing defining a viewing window through which the waste container is viewable. The system also includes a cover assembly coupled to the housing and may include a cover movably coupled to one of the housing and the chassis, and a pivot arm pivotably coupled to the cover. The cover assembly also includes an actuator pivotably coupled to the pivot arm and being slidable relative to the housing, the actuator configured to receive an input to move the cover to selectively occlude at least a portion of the window.

A tenth aspect includes a method of operating a medical waste collection system including an upper waste container. The method includes actuating, with the controller, the transfer valve to an open position to permit waste material to be transferred from the upper waste container to the lower waste container. The method also includes receiving, at the controller, fluid level signals from the at least one level sensor. The method also includes determining, with the controller, a change in the fluid level within the upper waste container based on the fluid level signals. The method also includes operating the vacuum source to draw suction on the lower waste container with the transfer valve in the open position to draw the waste material from the upper waste container into the lower waste container through the transfer valve based on the change in the fluid level during a dumping protocol being indicative of a clogged condition. The method also includes upper waste container decreasing to below a preset low fluid level.

An eleventh aspect includes a medical waste collection system for collecting waste material. The medical waste collection system includes an upper waste container. The system also includes a lower waste container. The system also includes at least one level sensor coupled to at least one of the upper waste container and the lower waste container. The system also includes a transfer valve coupled to the upper and lower waste containers and operable to permit the waste material to be transferred from the upper waste container to the lower waste container. The system also includes a vacuum source configured to provide independent suction to each of the upper waste container and the lower waste container. The system also includes a pump separate from the vacuum source and in fluid communication with the upper waste container. The system also includes a controller in electronic communication with the at least one level sensor, the transfer valve, and the pump. The controller is configured to determine a change of fluid level with the transfer valve in an open position based on level signals received from the at least one level sensor, and to operate the pump to direct air into the upper waste container based on the change in the fluid level during a dumping protocol being indicative of a clogged condition.

A twelfth aspect includes a method of operating a medical waste collection system including an upper waste container. The method includes actuating, with the controller, the transfer valve to an open position to permit waste material to be transferred from the upper waste container to the lower waste container. The method also includes receiving, at the controller, fluid level signals from the at least one level sensor. The method also includes determining, with the controller, a change in fluid level within the upper waste container based on the fluid level signals. The method also includes operating the pump to force air into the upper waste container with the transfer valve in the open position to urge the waste material into the lower waste container through the transfer valve based on the change of the fluid level during a dumping protocol being indicative of a clogged condition.

It should be appreciated that aspects, and their corresponding implementations, may be combined with one another. In particular, aspects directed to the light assembly may be included on any implementation of the cover assembly. Likewise, aspects directed to the dumping protocol may be included on the medical waste collection system in which any implementation of the light assembly and/or the cover assembly is provided.

### BRIEF DESCRIPTION OF THE DRAWINGS

FIG. 1 is a perspective view of a medical waste collection system.

FIG. 2 is a side elevation view of the medical waste collection system. Certain subcomponents are schematically represented.

FIG. 3 is a partial perspective view of the medical waste collection system with a light source assembly coupled to a waste container.

FIG. 4 is a sectional view of the light source assembly with the waste container and a fluid level sensor in a first state.

FIG. 5 is a sectional view of the light source assembly with the waste container and the fluid level sensor in a second state.

FIG. 6 is a perspective view of a first implementation of a cover assembly in a first state.

FIG. 7 is a perspective view of the first implementation of the cover assembly in a second state.

FIG. 8 is a perspective view of a second implementation of the cover assembly.

FIG. 9 is a perspective view of a third implementation of the cover assembly.

FIG. 10 is a partial perspective view of the third implementation of the cover assembly.

FIG. 11 is a partial perspective view of a fourth implementation of the cover assembly.

FIG. 12 is partial perspective view of the fourth implementation of the cover assembly.

FIG. 13 is a perspective view of a fifth implementation of the cover assembly in a first state.

FIG. 14 is a perspective view of the fifth implementation of the cover assembly in a second state.

FIG. 15A is a representative view of polarizers of the cover assembly of FIGS. 13 and 14 in the first state.

FIG. 15B is an elevation view of the polarizers of the cover assembly of FIGS. 13 and 14 in the first state.

FIG. 16A is a representative view of polarizers of the cover assembly of FIGS. 13 and 14 in the second state.

FIG. 16B is an elevation view of the polarizers of the cover assembly of FIGS. 13 and 14 in the second state.

FIG. 17 is an elevation view of a fifth implementation of the cover assembly.

FIG. 18 is a side elevation view of the medical waste collection system. Certain subcomponents are schematically represented.

FIG. 19 is a partial sectional view of the waste containers of the medical waste collection system.

FIG. 20 is a schematic view of certain electronic subcomponents of the medical waste collection system.

FIG. 21 is a flow chart of a method of performing protocols according to aspects of the present disclosure.

### DETAILED DESCRIPTION

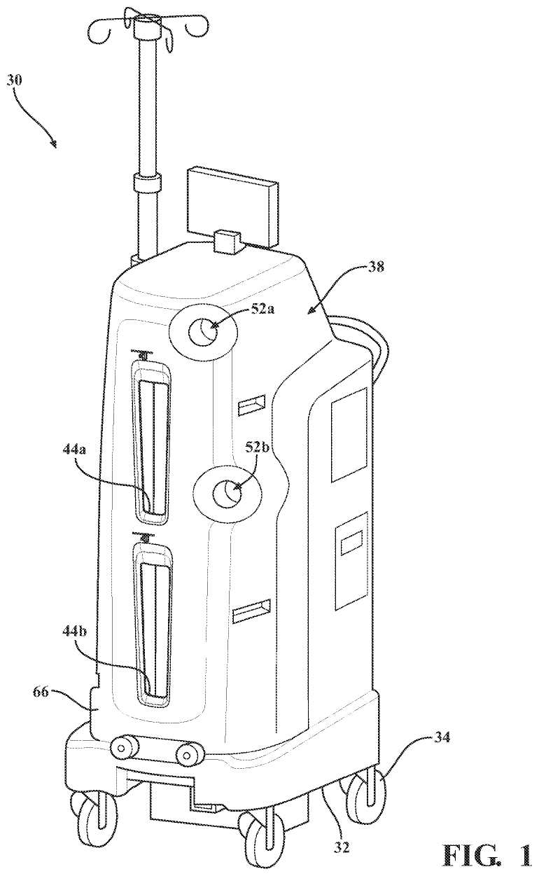

With reference to the drawings, where like numerals are used to designate like structure throughout the several views, a medical waste collection system for collecting waste materials is shown generally at 30 in FIG. 1. The medical waste collection system 30 collects waste material generated during medical procedures (e.g., surgical procedures) performed in a medical facility such as a hospital. The waste material may include bodily fluids, smoke, body tissues, irrigation liquids, or the like. The medical waste collection system 30 collects the waste material for later discharge. In particular, the medical waste collection system 30 collects and stores the waste material on-board to be off-loaded when removably coupled with a docking station. As such, the medical waste collection system 30 is capable of storing waste material from a series of different medical procedures during the course of a day or across several days, without requiring off-loading of the waste material.

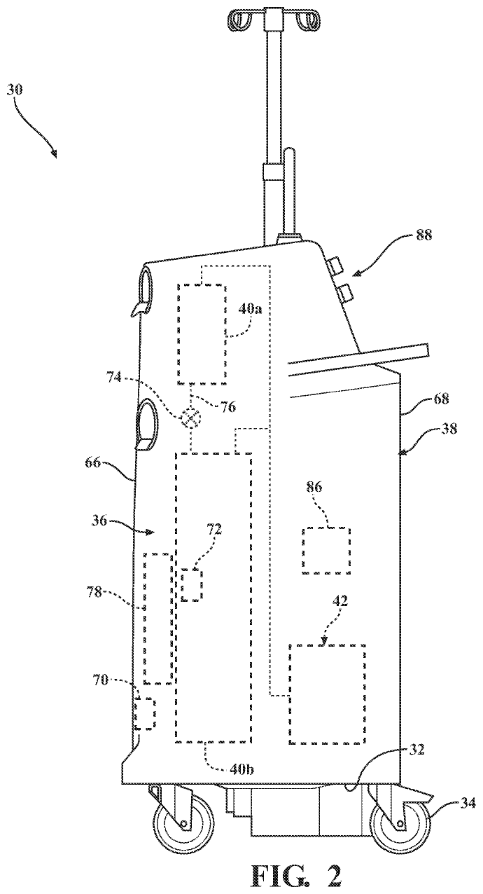

Referring to FIG. 2, the medical waste collection system 30 may include a base 32 and wheels 34 for moving the medical waste collection system 30 along a floor surface within the medical facility. The medical waste collection system 30 may further include a frame or chassis 36 extending upwardly from the base 32. The chassis 36 supports the components/subsystems of the medical waste collection system 30. A housing 38 or casing is coupled to the chassis 36 and defines an interior and described in further detail below. The housing 38 may be formed at least partially from a polymeric material, such as plastic casing shells. In other configurations, the chassis 36 and the housing 38 are completely integrated such that the chassis 36 and the housing 38 are indistinguishable.

The medical waste collection system 30 further includes one or more waste containers 40a, 40b to collect and store the waste material. The waste containers 40a, 40b may be disposed at least partially within the interior of the housing 38 and supported by the chassis 36. It is contemplated that the waste containers 40a, 40b may assume any shape that is suitable for containing the waste material. The waste containers 40a, 40b may include an upper waste container 40a and a lower waste container 40b, as depicted in FIG. 2. In other implementation, such as that disclosed in commonly-owned International Patent Publication No. WO 2020/210763, published Oct. 15, 2020, the contents of which are hereby incorporated by reference, a single waste container may be employed. The waste container(s) 40a, 40b may be formed of glass, suitable plastic materials, or other materials.

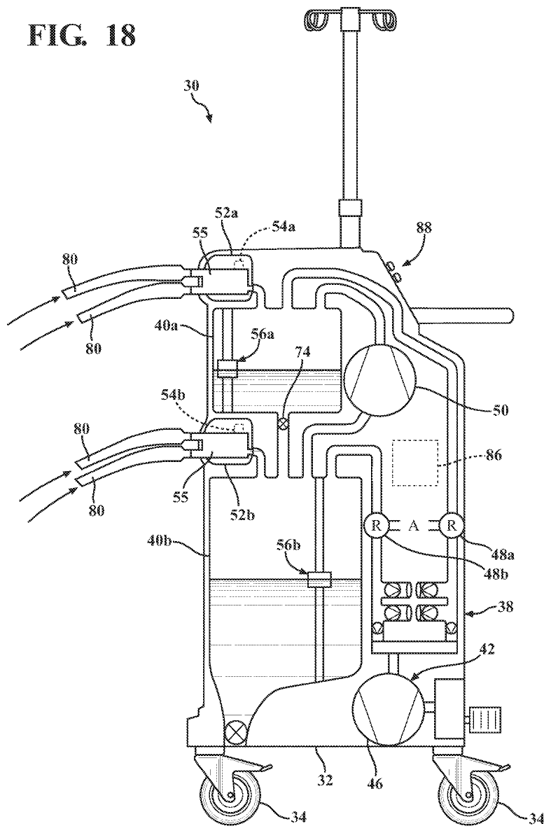

A vacuum source 42 may be supported on the base 32 and coupled to the chassis 36. The vacuum source 42 is configured to draw suction on the waste containers 40a, 40b through one or more vacuum lines (see FIG. 18). The vacuum source 42 may include a vacuum pump 46 and/or at least one vacuum regulator 48a, 48b configured to be in fluid communication with regulate a level of the suction drawn by the vacuum pump 46 on the waste containers 40a, 40b. More particularly, an upper vacuum regulator 48a may be operated to selectively establish fluid communication between the upper waste container 40a and atmospheric pressure A, or between the upper waste container 40a and the vacuum source 42; and a lower vacuum regulator 48b may be operated to selectively establish fluid communication between the lower waste container 40b and atmospheric pressure A, or between the lower waste container 40b and the vacuum source 42. Suitable construction and operation of several subsystems of the medical waste collection system 30 are disclosed in commonly-owned United States Patent Publication No. 2005/0171495, published Aug. 4, 2005, International Patent Publication No. WO 2007/070570, published Jun. 21, 2007, International Patent Publication No. WO 2014/066337, published May 1, 2014, International Patent Publication No. WO 2017/112684, published Jun. 29, 2017, and the aforementioned International Patent Publication No. WO 2020/210763, the entire contents of each being hereby incorporated by reference. In other configurations, the vacuum source 42 may be a separate unit that can be removably coupled to the medical waste collection system 30 to draw suction on the waste container 40. Suitable construction and operation of such a medical waste collection system 30 are disclosed in commonly owned U.S. Pat. No. 10,105,470, issued Oct. 23, 2018, the entire contents of which are hereby incorporated by reference.

Referring to FIGS. 1 and 2, the housing 38 includes a front portion 66 and a back portion 68 opposite the front portion 66. Each of the front and back portions 66, 68 of the housing 38 may include a single panel or multiple panels. The front portion 66 of the housing 38 has an exterior surface. The exterior surface of the front portion 66 may be adapted to generally face the surgical field during the medical procedure. The front portion 66 of the housing 38 may define one or more windows 44a, 44b to permit a user to view the waste containers 40a, 40b. With the waste containers 40a, 40b including a transparent material such as clear plastic, the user can see the waste material in the waste containers 40a, 40b through the windows 44a, 44b. The user can also view a level of waste material in the waste containers 40a, 40b. In the configuration illustrated in FIG. 1, the windows 44a, 44b are shown generally in the center (side to side) of the front portion 66 of the housing 38. However, it is contemplated that the windows 44a, 44b may be disposed off-center such that the windows 44a, 44b may be disposed to the left or right of the position of the windows 44a, 44b as viewed in FIG. 1.

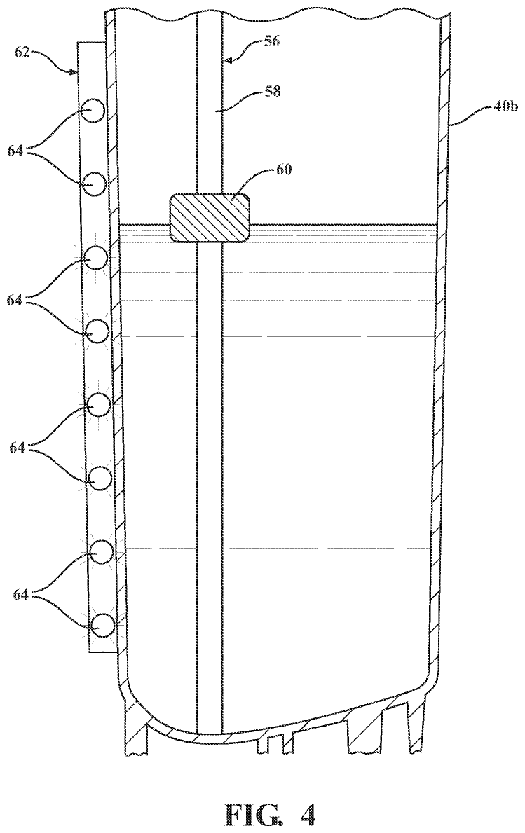

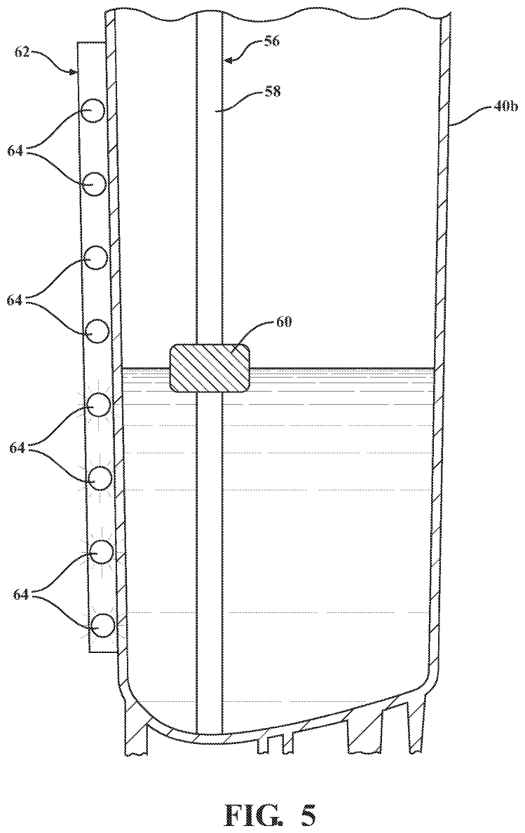

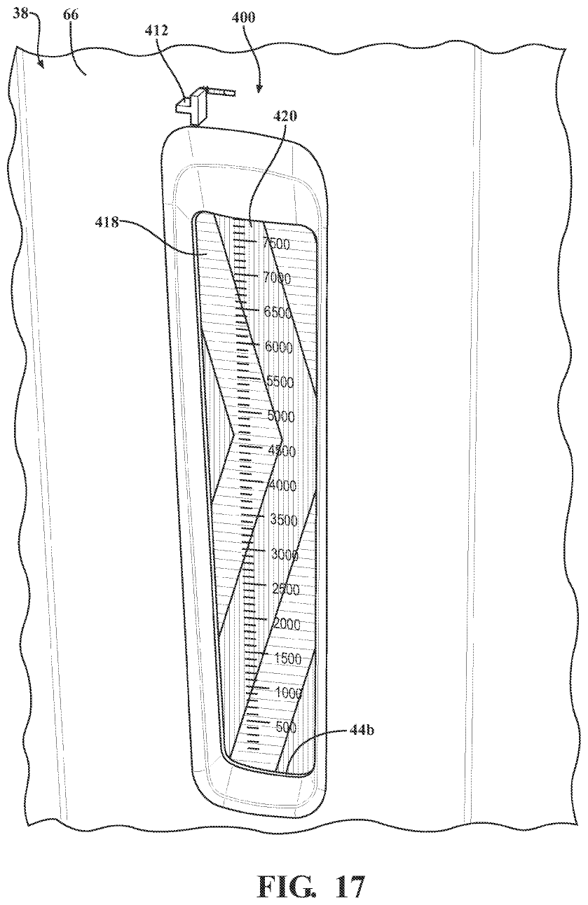

Referring to FIGS. 4 and 5, the medical waste collection system 30 may include a level sensor 56 coupled to the waste container 40b and configured to generate a signal indicative of a fluid level of the waste material disposed within the waste container 40b. The level sensor 56 may be realized as a liquid measuring system including a sensor rod 58 and floating element 60 as described in aforementioned International Patent Publication No. WO 2007/070570. With concurrent reference to FIG. 17, the sensor rod 58 may extend through and between the upper and lower waste containers 40a, 40b. In other configurations, each waste container 40a, 40b includes a discrete level sensor 56. Alternatively, it is contemplated that the level sensor 56 may include a different design that is capable of generating signals to indicate a fluid level disposed within the waste container 40b.

The medical waste collection system 30 may include a light source assembly 62 coupled to the waste container 40b. The light source assembly 62 may include a plurality of light sources 64. Each of the light sources 64 may be configured to be selectively operable to illuminate an interior of the waste container 40b. The plurality of light sources 64 may be spaced apart from one another vertically with respect to gravity. The light sources 64 illustrated in FIGS. 4 and 5 are spaced apart from one another equidistantly. However, it is contemplated that the light sources 64 may be disposed at different distances relative to each other such that a first distance between a first light source and a second light source is different than a second distance between the second light source and a third light source, and so on. Each of the light sources 64 may be a light emitting diode (LED), a bulb, a lamp, or another like device configured to emit visible light.

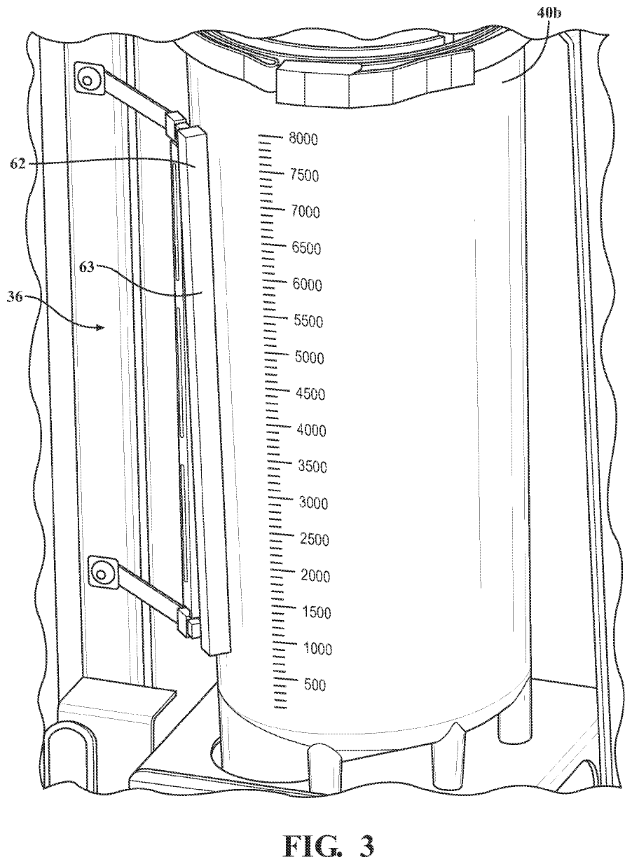

FIG. 3 shows the light source assembly 62 being a light strip having a substrate 63. The plurality of light sources 64 are coupled to the substrate 63. The substrate 63 may be coupled to the chassis 36 via support arms to space the substrate 63 away from the chassis 36 and against the waste container 40b. In other configurations, the substrate 63 may be coupled directly to the waste container 40b without additional attachment to the chassis 36 or housing 38. Such an arrangement may mitigate light from bleeding out away from the waste container 40b. For example, the light sources 64 in FIGS. 4 and 5 are shown coupled to a sidewall of the waste container 40b. In other configurations, the light source assembly 62 may include multiple substrates spaced apart vertically including one or more light sources 64. In still another variant, the light strip may be disposed adjacent to a side of the window 44b of the housing 38, and the light source assembly 62 may further include a second light strip disposed adjacent to an opposing side of the viewing window 44b. It is also contemplated that one or more of the light sources 64 may be coupled to a bottom of the waste container 40b.

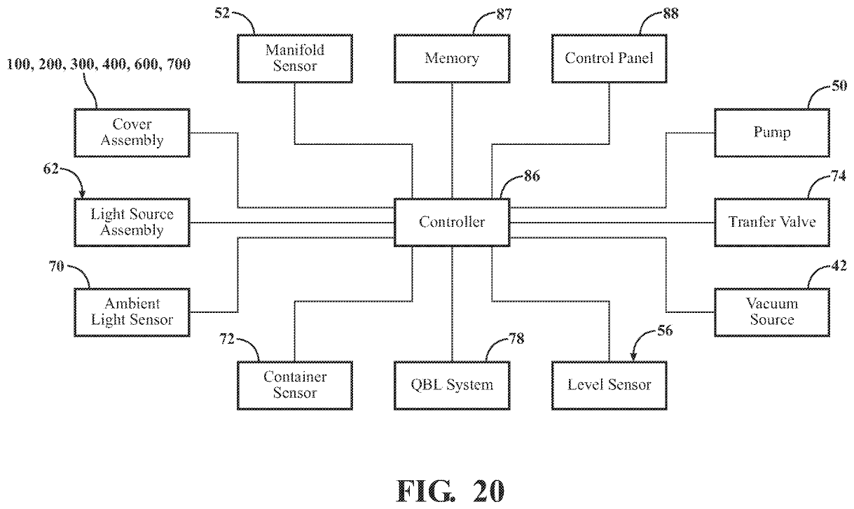

The medical waste collection system 30 includes a controller 86 (shown schematically in FIGS. 2 and 20). The controller 86 may be a plurality of sub-controllers, each including one or more microprocessors, processors, systems on a chip, etc., to operate certain features of the medical waste collection system 30. The controller 86 is in electronic communication with the level sensor 56 and the light source assembly 62. The controller 86 is configured to activate or operate the light sources 64, and more particularly selectively and individually operate each of the light sources 64. In manners to be described, the controller is configured to control, activate, or operate fewer than all of the light sources 64 based on the signal from the level sensor 56 such that a portion of the interior of the waste container 40b disposed beneath the fluid level of the waste material is illuminated. Said differently, the controller 86 may be configured to not operate one or more of the light sources 64 disposed above the fluid level of the waste material in the waste container 40b based on the signal. By illuminating the waste material in the waste container 40b, an empty portion of the waste container 40b disposed above the waste material remains relatively dark. In such instances, a potentially soiled sidewall of the waste container 40a, 40b is less visible.

An on-board control panel 88 may be configured to generate signals to and receive signals from the controller 86 to permit the user to selectively operate the light source assembly, the vacuum source 42, and other systems of the medical waste collection system 30. In the configuration illustrated in FIG. 2, the control panel 88 is coupled to the top portion of the housing 38. The control panel 88 may include one or more user interfaces such as knobs, dials, touch screen inputs, or the like, in communication with the controller 86, to allow the user to establish the desired vacuum levels in the waste containers 40a, 40b. The control panel 88 may also indicate to the user data relating to a suction or vacuum level on the waste containers 40a, 40b. Suitable operation of a medical waste collection system 30 to control vacuum levels in the waste containers 40a, 40b is disclosed in commonly-owned U.S. Pat. No. 7,621,898, granted Nov. 24, 2009, the entire contents of which are hereby incorporated by reference.

As mentioned, the waste material on-board is off-loaded by coupling the medical waste collection system 30 with the docking station, which also performing a cleaning cycle of the waste container(s) 40a, 40b. Despite the cleaning cycle, the inner walls of the waste container 40b may at least somewhat residually soiled. Additionally or alternatively, the inner walls may become soiled during instances in which high suction is being drawn on the waste container 40a, 40b (e.g., owing to splashing or sloshing). Often, portions of the soiled wall above the fluid level within the waste container(s) 40a, 40b are particularly visible with light passing therethrough. Therefore, to avoid the soiled walls of the waste container 40b being unduly visible, the controller 86 may operate the light source assembly 62 to only generate light with the light sources 64 disposed beneath the surface of the waste material. As a result, the user may clearly see fluid levels, colors, and consistencies of the waste material within the waste container 40b, without drawing attention to the empty portions of the waste container 40b.

The controller 86 may include or be in electronic communication with memory 87 storing data associating a vertical position of each of the plurality of light sources 64 to a corresponding predefined fluid level within the waste container 40b. The controller 86 may be configured to receive the signal from the level sensor 56 and determine the fluid level of waste material disposed within the waste container 40b based on the signal. The controller 86 then compares the data accessed from the memory 87 with the determined fluid level and operates fewer than all of the light sources 64 based on the comparison. In some instances where the fluid level is disposed above all the light sources 64, the controller 86 may be configured to operate all of the light sources 64. The light sources 64 may be configured to emit light in variable light intensity, light colors, and/or light patterns. The memory 87 may store data further associating the predefined fluid levels with predefined light intensities, light colors, and light patterns. The controller 86 may be configured to operate fewer than all of the light sources 64 at the predefined light intensities based on the signal.

Instead of operating the light source assembly 62 to only generate light with light sources 64 disposed beneath the surface of the waste material, a moveable cover (not shown) disposed between the waste container 40b and the housing 38 may be employed to shield a portion of the waste container 40b disposed above the waste material. The moveable cover may be configured as a door, a slide, a ribbon, a drape, polarized lenses, or another shielding member intended to reduce visibility of the portion of the waste container 40b disposed above the waste material. In some configurations, the moveable cover may be raised and lowered by an actuator of a cover assembly 100, 200, 300, 400, 600, 700 that receives an input from the user, or by a motor operatively coupled to the moveable cover. The memory 87 may store data further associating the predefined fluid levels of waste material with predefined positions of the moveable cover. The controller 86 may be configured to operate the actuator to adjust a position of the moveable cover at the predefined positions based on the signal from the level sensor such that the moveable cover may be raised and lowered automatically in response to changing fluid waste level in the waste container 40b. The cover assembly 100, 200, 300, 400, 600, 700 is described in greater detail further below.

It is also contemplated that any number or combination of light sources 64 disposed above or below the fluid waste level could be illuminated. In one configuration, light sources 64 disposed above the fluid waste level may be automatically dimmed relative to light sources 64 disposed below the fluid level. For instance, the controller 86 may be configured to operate light sources 64 disposed above the fluid waste level at a first intensity and light sources 64 disposed beneath the fluid waste level at a second intensity greater than the first intensity. In some configurations the first intensity may be so low as to approximate no light being generated.

Additional configurations of controlling the light source assembly 62 are contemplated. The controller 86 may be configured to operate light sources 64 disposed farthest from the fluid waste level at a greater intensity than light sources 64 disposed closer to the fluid waste level. The controller 86 may be configured to operate the light sources 64 disposed above the fluid level as a dark color and light sources 64 disposed beneath the fluid level as a lighter color. The controller 86 may be configured to operate the light sources 64 in a variety of different patterns such that the light sources 64 generate light in a sequence, e.g., light source illuminate in order from bottom to top. Finally, the controller 86 may be configured to operate the light sources 64 so that every other light source 64 is illuminated in a blinking fashion. Other patterns are contemplated.

In the configuration illustrated in FIGS. 4 and 5, changes in fluid level and light source illumination are shown. The light source assembly 62 includes eight light sources 64 spaced apart vertically relative to the waste container. In FIG. 4, the waste material level is disposed between the sixth and the seventh light source 64 from the bottom. The controller 86 receives a signal from the level sensor 56 and compares the signal with the data from memory to determine which light sources 64 are disposed beneath the fluid level. The controller 86 operates one or more of the light sources 64 beneath the waste material level to illuminate only the waste material and not the portion of the waste container 40b above the waste material. In FIG. 5, the waste material level is disposed between the fourth and fifth light source 64 from the bottom. The controller 86 receives a signal from the level sensor 56 and compares the signal with the data from memory to determine which light sources 64 are disposed beneath the fluid level. The controller 86 operates one or more of the light sources 64 beneath the waste material level to illuminate only the waste material and not the portion of the waste container 40b above the waste material. While the light source assembly 62 in the configurations illustrated in FIGS. 4 and 5 include eight light sources 64, it is appreciated that the light source assembly 62 may include seven or fewer light sources 64. It is also contemplated that the light source assembly 62 may include nine or more light sources 64.

As mentioned, the light sources 64 disposed beneath the waste material level may be operated by the controller 86 to illuminate the waste container 40b. In other configurations, one or more of the light sources 64 disposed immediately beneath the fluid level are not operated, and the fluid is illuminated with less than all the light sources 64. In particular, the controller 86 may be configured to operate one or more of the first n−1 light sources 64 from the bottom that are disposed beneath the waste material level, where n is the number of light sources 64 disposed beneath the fluid level. In such a configuration, the nearest light source 64 to the fluid level that is beneath the fluid level may not be operated to illuminate the waste container 40b so that in instances where the waste material is agitated and the fluid level momentarily fluctuates, light will not bleed into the portion of the waste container 40b above the fluid level.

In some configurations, the user interface of the control panel 88 may be configured to receive an input from the user to control the light source assembly 72 in any number of the aforementioned and other configurations. For example, the user may select full illumination of the waste container 40b such that the controller 86 operates all of the plurality of light sources 64 regardless of the fluid level. Another example includes the user selecting a light characteristic such as a selected light color and/or a selected light intensity. The controller 86 may correspondingly operate fewer than all the plurality of light sources 64 at the selected light color, the selected light intensity, or other light characteristic.

The medical waste collection system 30 may include an ambient light sensor 70 configured to generate a signal indicative of an intensity of ambient light. The controller 86 may be in electronic communication with the ambient light sensor 70. The controller 86 may be configured to operate the light sources 64 to illuminate an interior of the waste container 40b at a determined intensity based on the signal received from the ambient light sensor 70. In some configurations, the brightness of light sources 64 may be increased when the ambient light sensor 70 detects a bright light around the medical waste collection system 30. In other configurations, the brightness when the ambient light sensor 70 may be decreased when the ambient light sensor 70 detects dim light around the medical waste collection system 30. Medical procedures using the medical waste collection system 30 are often conducted in procedure rooms where the environment surrounding the patient is dimly lit. For instance, in gastrointestinal endoscopic procedures, it is important that the procedure room remain dark or dimly lit so that an endoscope (not shown) used during the procedure can effectively capture images of the procedure site for later analysis and display them on a monitor (not shown) in the procedure room without light in the procedure room (e.g., from the medical waste collection system 30) producing a glare on the monitor.

The controller 86 may be configured to operate the light sources 64 to be brighter in response to the signal being indicative that the intensity of the ambient light is above an ambient light threshold. The controller 86 may also be configured to operate the light sources 64 for the determined intensity to approximate the intensity of the ambient light. The medical waste collection system 30 may include a container light sensor 72 coupled to the waste container 40b. The memory 87 may store data including a predefined color intensity for image capture of the interior of the waste container with a camera for quantitative blood loss (QBL) analysis. The controller 86 may be further configured to operate the light sources 64 for the determined intensity to approximate the predefined color intensity based on feedback from the container light sensor 72. The predefined color intensity may include a whiteness color value. Operating the light sources 64 to approximate the whiteness color value allows for more accurate readings during QBL analysis. Suitable construction and operation of a QBL system in which a camera is positioned adjacent to the waste container 40 to capture images of the waste material is disclosed in commonly-owned International Application No. PCT/US2023/025603, filed Jun. 16, 2023, the entire contents of which being hereby incorporated by reference.

The controller 86 may receive QBL data from a QBL system 78, shown schematically in FIG. 2. The QBL data may be indicative of a blood concentration within the waste material. The controller 86 may operate the vacuum source to draw medical waste into the waste container 40b. The controller 86 may receive fluid level signals from the level sensor 56 and determine a fluid volume of the medical waste within the waste container 40b. The controller 86 may then determine lost blood volume based on the blood concentration and the fluid volume. The controller 86 may operate the light source 64 to indicate any number of conditions or statuses based on the QBL analysis. For example, the light sources 64 may be selectively operated in pattern, color, or intensity to indicate a warning condition in which the lost blood volume deviates beyond a predetermined threshold. For another example, the light sources 64 may be selectively operated in pattern, color, or intensity to indicate an error condition in which the lost blood volume is greater than the fluid volume.

With continued reference to FIG. 2, the light sources 64 may be used to indicate a clogged condition or an insufficient volume condition of the medical waste collection system 30. A transfer valve 74 may be coupled to the first and second waste containers 40a, 40b and the controller 86. The transfer valve 74 may be disposed on a dump line 76 and configured to be opened to permit dumping of the waste material from the upper waste container 40a to the lower waste container 40b. In other instances, the transfer valve may be built into one of the waste containers 40a, 40b. In such an implementation, the transfer valve may be larger in size than when disposed on the dump line 76. The controller 86 may operate the transfer valve 74 to initiate a dumping protocol to be described. As also to be described in greater detail, the controller 86 receives signals from the level sensor(s) 56 indicative of a corresponding change in the fluid level in at least one of the upper and lower waste containers 40a, 40b. The controller 86 then operates the light source 64 to indicate a clogged condition in which a rate of increase of the fluid level in the second waste container 40b is below a preset fluid transfer rate. The controller 86 may be configured to operate the light source 64 in one of a predefined color and a blinking sequence to indicate the clogged condition or an insufficient volume condition. The insufficient volume condition is based on an anticipated rate of increase of the fluid level being expected to be greater than a remaining available volume of the waste container 40b in less than a predetermined period. The anticipated rate of increase may be based on the rate of increase of the fluid level up to that point of the medical procedure.

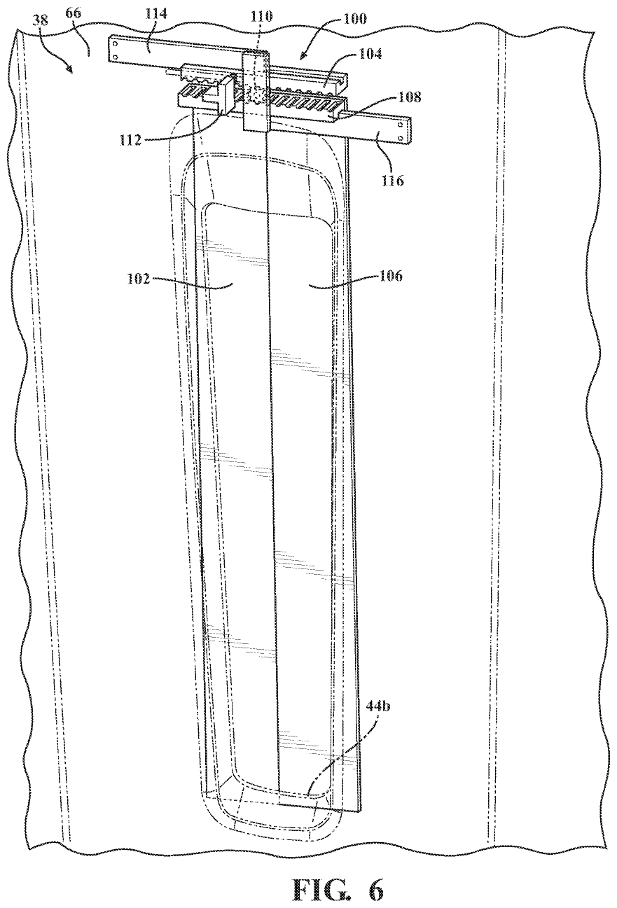

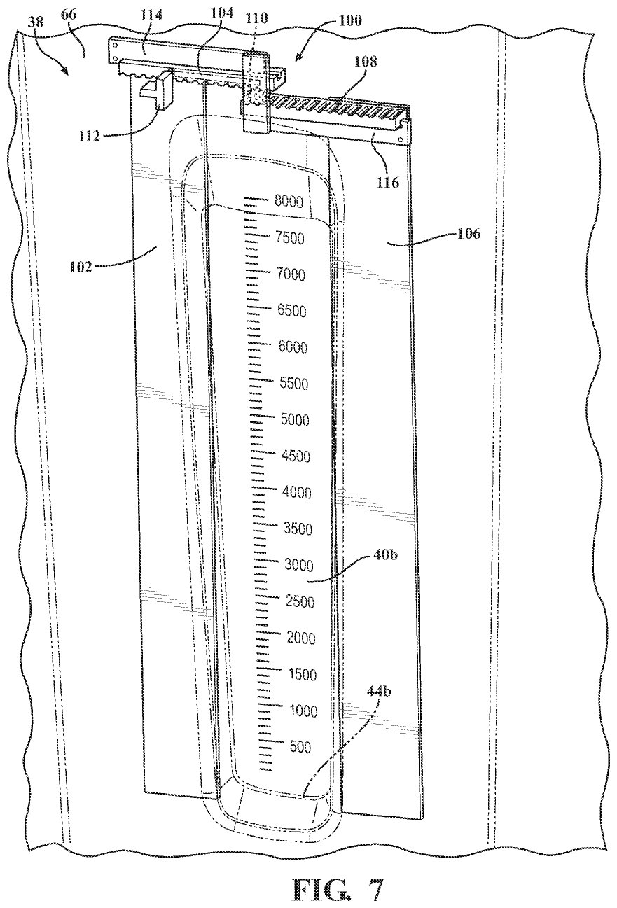

In addition or as an alternative to using the light assembly 62 to limit visualization of portions of the waste containers 40a, 40b, it may be desirable to obscure portions of the waste containers 40a, 40b with a cover assembly 100, 200, 300, 400, 600, 700. As mentioned, medical waste collection systems 30 are generally portable for moving throughout the health care facility. To support portability, the medical waste collection system 30 is designed to be compact and mitigate empty space within the housing 38. Consequently, the cover assembly 100, 200, 300, 400, 600, 700 of the present disclosure is correspondingly compact and actuatable in an intuitive and ergonomic manner. Referring to FIGS. 6 and 7, a first implementation of a cover assembly 100 of the medical waste collection system 30 is shown. The front portion 66 of the housing 38 is shown in phantom to better illustrate the cover assembly 100. The cover assembly 100 may be used for selectively permitting a user to view the waste containers 40a, 40b, through the windows 44a, 44b. In the configuration illustrated in FIGS. 6 and 7 the cover assembly 100 is used to selectively permit a user to view the lower waste container 40b.

The cover assembly 100 may be coupled to the chassis 36 or the housing 38. The cover assembly 100 includes a first cover 102 having a first rack 104 and a second cover 106 having a second rack 108. The first and second racks 104, 108 may each include a plurality of rack teeth. A pinion 110 may be disposed between the first and second covers 102, 106 and have pinion teeth disposed in meshing engagement with the first and second racks 104, 108 such that movement of first cover 102 in one direction causes movement of the second cover 106 in an opposite direction.

In other configurations where at least one of the pinion 110 and the racks 104, 108 do not include teeth, there may be sufficient friction between the pinion 110 and the racks 104, 108 such that movement of first cover 102 in one direction causes movement of the second cover 106 in an opposite direction. In such a configuration, the racks 104, 108 may engage the pinion 110 with the pinion 110 configured as a roller. The racks 104, 108 may include generally planar surfaces abutting the roller and the racks 104, 108 may be disposed on either side of the roller such that movement of first cover 102 in one direction causes movement of the second cover 106 in an opposite direction.

In another configuration, the cover assembly 100 may comprise a belt (not shown) that may be looped around two pulleys. The racks 104, 108 may be coupled to the belt between the pulleys and with each rack 104, 108 disposed on a different side of the loop (i.e., above or below the pulleys) such that movement of first cover 102 in one direction causes movement of the second cover 106 in an opposite direction. The pinion 110 may be disposed between the pulleys and abutting the belt such that rotation of the pinion 110 may drive the belt. In other configurations, one of the pulleys may comprise the pinion 110.

An actuator 112 (e.g., a switch or a lever) may be coupled to the first cover 102 and configured to receive an input to move the first and second covers 102, 106 towards one another to selectively occlude at least a portion of the window 44b. The actuator 112 may include a single actuation slider to operate the cover assembly 100 with one input. In other words, a user may grasp the actuator 112 with one hand and close the cover assembly 100 with one hand. Employing a rack and pinion design that moves two covers 102, 106 simultaneously with a single input allows the overall movement of the covers 102, 106 and the actuator 112 to be reduced which requires less of an overall footprint of the cover assembly 100 within the housing 38 and around the waste containers 40a, 40b than if a single cover were employed. Furthermore, using a single actuator 112 is less cumbersome than using multiple actuators to move the covers 102, 106 independently.

The cover assembly 100 may be moveable between a first state (FIG. 6) where the first and second covers 102, 106 collectively occlude the window 44b and a second state (FIG. 7) where the first and second covers 102, 106 are spaced from one another such that the window 44b is not occluded. The actuator 112 may be selectively moveable between a first position (FIG. 6) and a second position (FIG. 7). In the first position, the cover assembly 100 is in the first state. In the second position, the cover assembly 100 is in the second state.

The cover assembly 100 may include first and second rails 114, 116 coupled to at least one of the chassis 36 and the housing 38 and configured to support movement of the first and second covers 102, 106, respectively. The rails 114, 116 may be fixed to the chassis 36 and/or the housing 38 and configured to guide the first and second covers 102, 106 when the actuator 112 is moved by the user. The pinion 110 may be coupled to the chassis 36 and/or the housing 38 and configured to rotate about a pivot that is fixed relative to the chassis 36 and the housing 38. In some configurations the pinion 110 may be coupled to at least one of the first and second rails 114, 116. In the illustrated configuration, portions of each rail 114, 116 are received in a recess of the corresponding rack 104, 108 and configured for relative sliding movement. The recess may be configured as a dovetail design to constrain relative movement to be slidable. In other configurations, the recess may comprise another shape to permit sliding between the racks 104, 108 and the rails 114, 116. The rollers may be attached to the racks 104, 108 and the rails 114, 116 may comprise a track to receive the rollers. The rollers may rotate relative to the track and the racks 104, 108 such that rotation of the roller causes the roller to roll along the track, moving the racks 104, 108 and the covers 102, 106 along the rails 114, 116.

In another configuration, the actuator 112 may be coupled to the pinion 110 instead of one of the covers 102, 106. In such a configuration, the actuator 112 may include a knob that is rotatable with the pinion 110 relative to the chassis 36 and the housing 38. A user may grasp the knob and provide a single input to rotate the pinion 110 and, due to the meshing engagement of the pinion 110 with the first and second racks 104, 108, move the first and second covers 102, 106 simultaneously between the first and second states to selectively open and close the covers 102, 106.

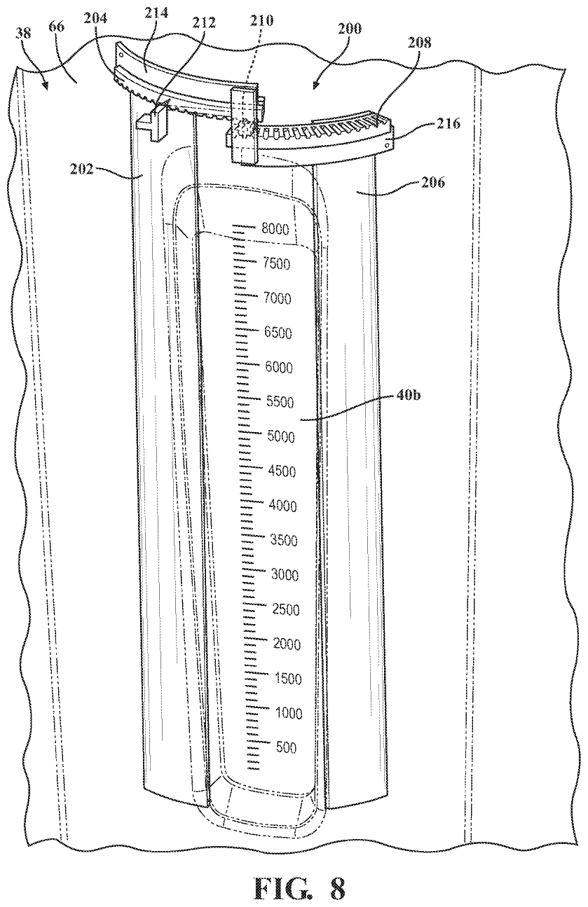

In a second implementation of the cover assembly 200 shown in FIG. 8, the first and second racks 204, 208 may be curved and contoured to an outer surface of the waste container 40b. In this manner, the first and second covers 202, 206 may be configured to arcuately move around the outer surface of the waste container 40b. In some configurations of the curved first and second racks 204, 208, the pinion 210 may have a conical or beveled shape to support the arcuate motion of the first and second racks 204, 208. The first and second covers 202, 206 may be supported by first and second rails 214, 216, respectively that are also curved to guide the covers 202, 206 arcuately.

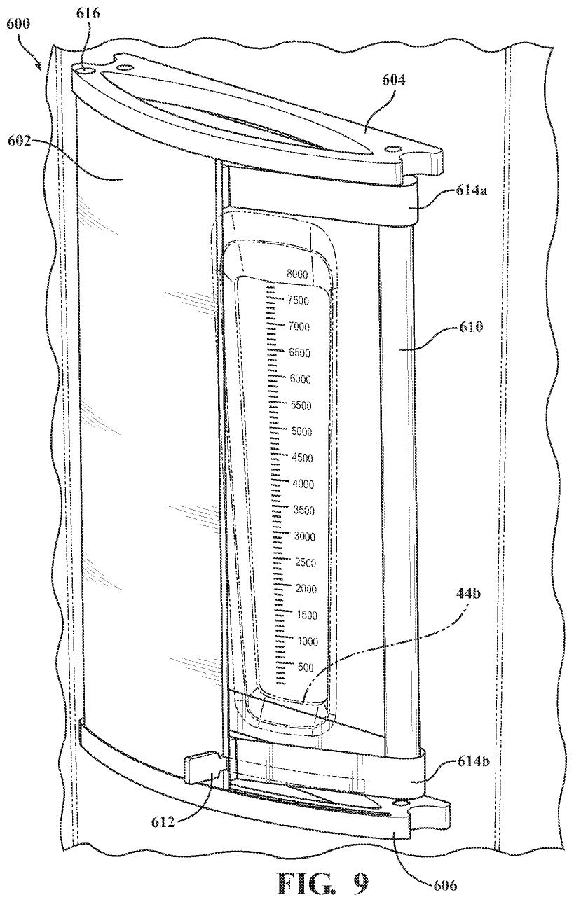

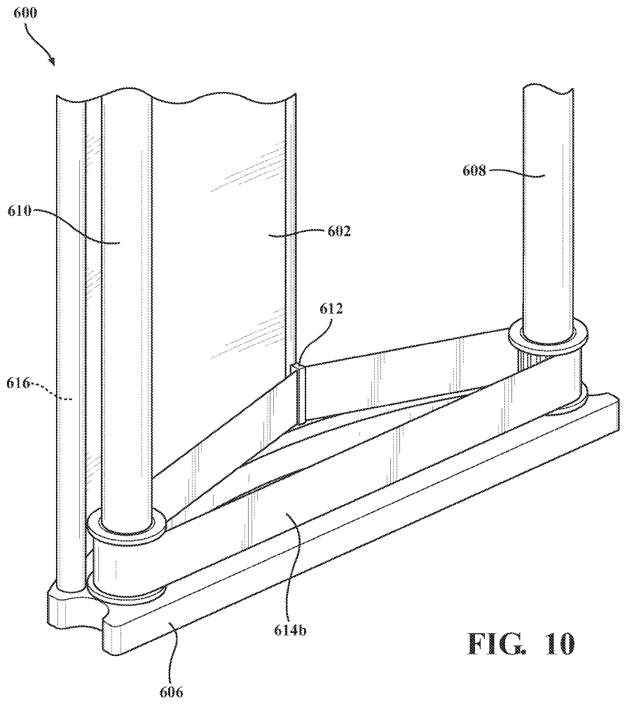

Referring to FIGS. 9 and 10, a third implementation of a cover assembly 600 of the medical waste collection system 30 is shown. The cover assembly 600 may be coupled to the chassis 36 or the housing 38. The cover assembly 600 includes a cover 602, a top guide 604, and a bottom guide 606. The cover 602 may be flexible and the guides 604, 606 may be configured to maintain a curved shape of the cover 602 as the cover 602 occludes the window 44b. The curved shape may conform to a shape of the waste containers 40a, 40b or the housing 38. In some configurations a single guide is used. The cover assembly 600 may comprise support rods 608, 610 rotatably coupled to both the top and bottom guides 604, 606. The support rods 608, 610 may be spaced apart from each other on either side of the window 44b. A belt 614 is coupled to the cover 602 and wrapped around the support rods 608, 610. The belt 614 is moveable with the cover 602 around the support rods 608, 610 to selectively occlude the window 44b. In some configurations, two belts 614a, 614b are used. The belts 614a, 614b are spaced apart from one another and prevent binding of the cover 602 in the guides 604, 606. In some configurations, the belt 614a, 614b has teeth. Use of a belt may be preferrable in some instances to reduce noise of the cover assembly 600 when the cover 602 moves to occlude or reveal the window 44b.

An actuator 612 (e.g., a switch or a lever) may be coupled to the cover 602 and/or the belts 614a, 614b and configured to receive an input to move the cover 602 relative the top and bottom guides 604, 606 to selectively occlude at least a portion of the window 44b. The actuator 612 may include a single actuation slider to operate the cover assembly 600 with one input. In other words, a user may grasp the actuator 612 with one hand and close the cover assembly 600 with one hand.

The cover assembly 600 may comprise a winding rod 616 positioned adjacent one of the support rods 608, 610 to maintain a portion of the cover 602 that does not occlude the window 44b. The winding rod 616 may be spring loaded (e.g., by a torsion spring) to wind up the cover 602 when the cover is not held in place. In other configurations, the winding rod 616 may be tuned to be strong enough to wind up slack in the cover 602 and weak enough so as to not rotate the belts 614a, 614b. In some cases, a hook may be coupled to the cover 602, the actuator 612, and/or the belt 614a, 614b to grab onto a portion of the housing 38 or chassis 36. The hook may be used to maintain a position of the cover 602 relative the housing 38 e.g., the hook may maintain the cover 602 in such a position that the cover 602 occludes the window 44b. The winding rod 616 allows the cover 602 to take up less space inside the housing 38 when the cover 602 is not occluding the window 44b, which reduces the overall footprint of the cover assembly 600.

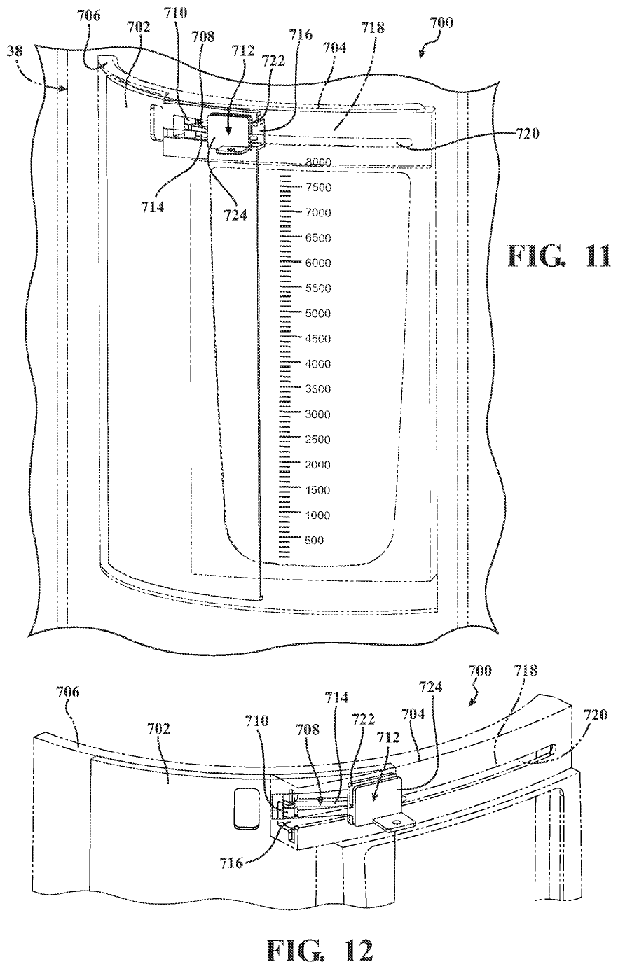

Referring to FIGS. 11 and 12, a fourth implementation of the cover assembly 700 of the medical waste collection system 30 is shown. The cover assembly 700 may be coupled to the chassis 36 or the housing 38. The cover assembly 700 includes a cover 702 and a guide 704. The cover 702 and the guide 704 may be curved to conform to a shape of the waste containers 40a, 40b or the housing 38. The cover 702 may comprise a rigid plastic and the guide 704 may define a channel 706 to receive the cover 702 and constrain the position of the cover 702 as the cover 702 moves relative the housing 38.

The cover assembly 700 also includes a pivot arm 708 or linkage pivotably coupled to the cover 702. The pivot arm 708 may include a first pivot portion 710 pivotably coupled to the cover. The pivot arm 708 may include a second pivot portion (not shown) pivotably coupled to an actuator 712 (e.g., a switch or a lever). The pivot arm 708 may also include an elongated portion 714 extending between the first and second pivot portions. In some configurations, the elongated portion 714 comprises an arcuate shape. In some configurations, the cover 702 may define a recess 716 to receive at least the first pivot portion 710. The arcuate shape of the elongated portion 714 and the recess 716 may be employed collectively or independently to mitigate the risk of binding between the cover 702 and the actuator 712.

As noted above, the actuator 712 is pivotably coupled to the pivot arm 708 and is slidable relative to the housing 38. The housing 38 may comprise a track 718 that defines a slot 720 to receive the actuator 712. The actuator 712 is configured to receive an input to move within the slot 720 and move the cover 702 to selectively occlude at least a portion of the window 44b. The actuator 712 may comprise a tab graspable by the user to move the actuator 712 along the track 718 within the slot 720. The tab may comprise a first flange 722 disposed on a first side of the track 718 facing the waste container 40a and being pivotably coupled to the pivot arm 708. The tab may also comprise a second flange 724 disposed on a second side of the track 718 facing away from the waste container 40a. The first and second flanges 722, 724 cooperate to maintain a portion of the tab within the slot 720.

In configurations where the cover 702 comprises a curved shape, the guide 704 may subtend a first arc having a first center point. The track 718 may extend linearly or the track may subtend a second arc having a second center point different from the first center point. The pivot arm 708 permits the actuator 712 to slide linearly or along the second arc different than the arc subtended by the cover 702. In other words, the pivot arm 708 allows the orientation of the cover 702 to change relative to the actuator 712 as the user moves the actuator 712. This configuration allows the track 718 of the housing 38 conform to shapes different than the shape of the cover 702.

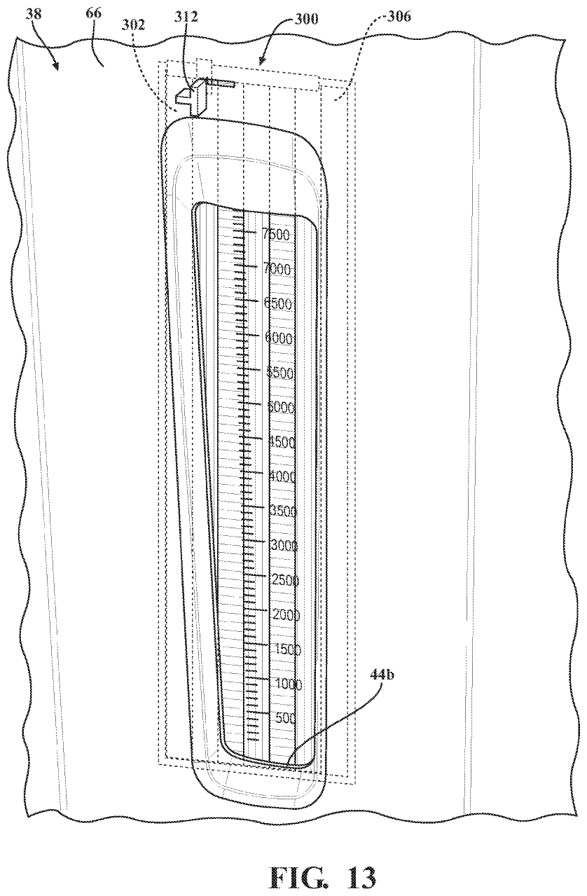

Referring to FIGS. 13-16B, a fifth implementation of a cover assembly 300 of the medical waste collection system 30 is shown. The cover assembly 300 is coupled to the housing 38 and/or the chassis 36. The cover assembly 300 may include a first cover 302 comprising a first polarizer and a second cover 306 moveable relative to the first cover comprising a second polarizer. The second polarizer has a different polarizing orientation than the first polarizer. An actuator 312 may be coupled to the first cover 302 or the second cover 306 and configured to receive an input to move the first cover 302 and the second cover 306 relative to one another to selectively reduce light being transmitted through the cover assembly 300 based on misalignment of the first and second polarizers. The actuator 312 may include a single actuation slider to operate the cover assembly 300 with a single input. In other words, a user may grasp the actuator 312 with one hand and operate the cover assembly 300 with one hand. In one configuration, one of the first and second covers 302, 306 is fixed to the housing 38 and/or the chassis 36 and the other of the first and second covers 302, 306 is moveable in response to operation of the actuator 312.

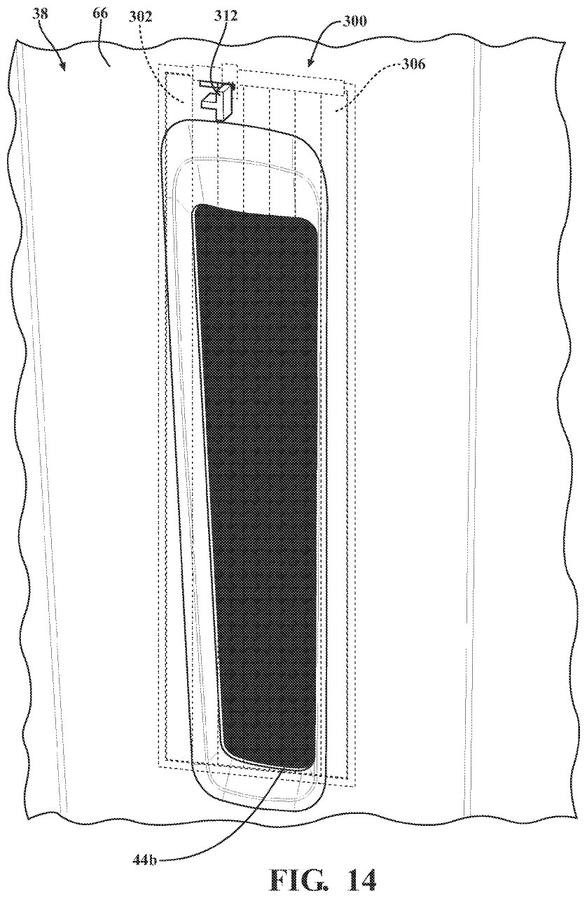

The first and second polarizers each include at least a first segment 318 and a second segment 320. The polarizing orientations of the first segments 318 are different than the second segments 320. The cover assembly 300 is operable between a first state (FIG. 13) in which the first segments 318 of the first and second polarizers are aligned to permit a first transmission of light, and a second state (FIG. 14) in which the first segment 318 of the first polarizer is aligned with the second segment 320 of the second polarizer to permit a second transmission of light that is less than the first transmission of light. The first transmission of light may be approximately 45-50% percent of ambient light. The second transmission of light may approximate zero. Other reductions in the transmission of light in each of the first state and/or the second state are contemplated.

In the first state, even with the reduced transmission of light, the waste container 40b is still visible through the window 44b. In the second state, the waste container 40b may not be viewable through the window. While the first and second covers 302, 306 in the configuration illustrated in FIGS. 13-16B each include three first segments 318 and three second segments 320, it is contemplated that the first and second covers 302, 306 may include any number of segments 318, 320 so long as at least one first segment 318 of one of the first and second covers 302, 306 aligns with a first segment 318 of the other of the first and second covers 302, 306 in the first state and at least one first segment 318 of one of the first and second covers 302, 306 aligns with a second segment 320 of the other of the first and second covers 302, 306 in the second state.

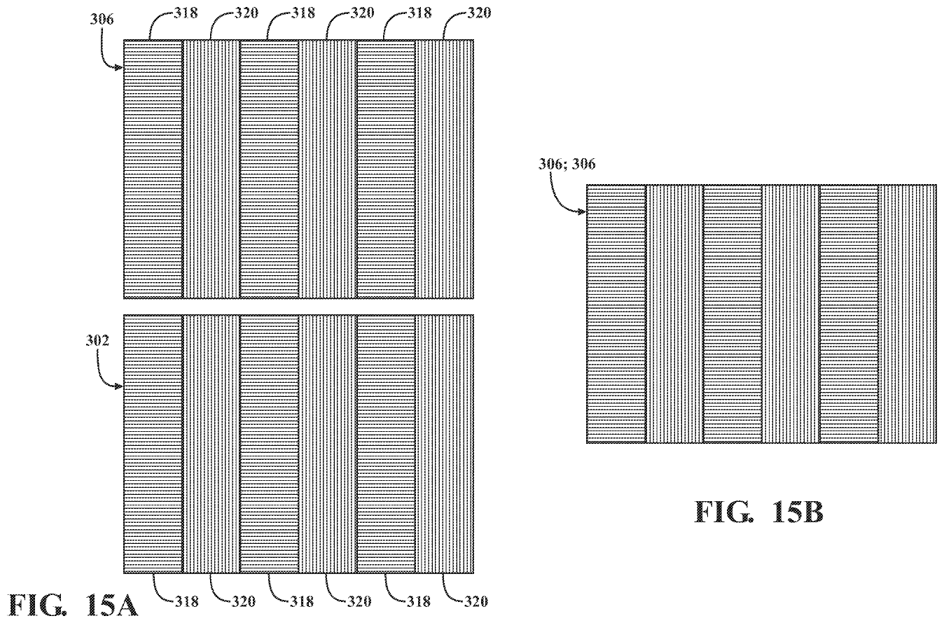

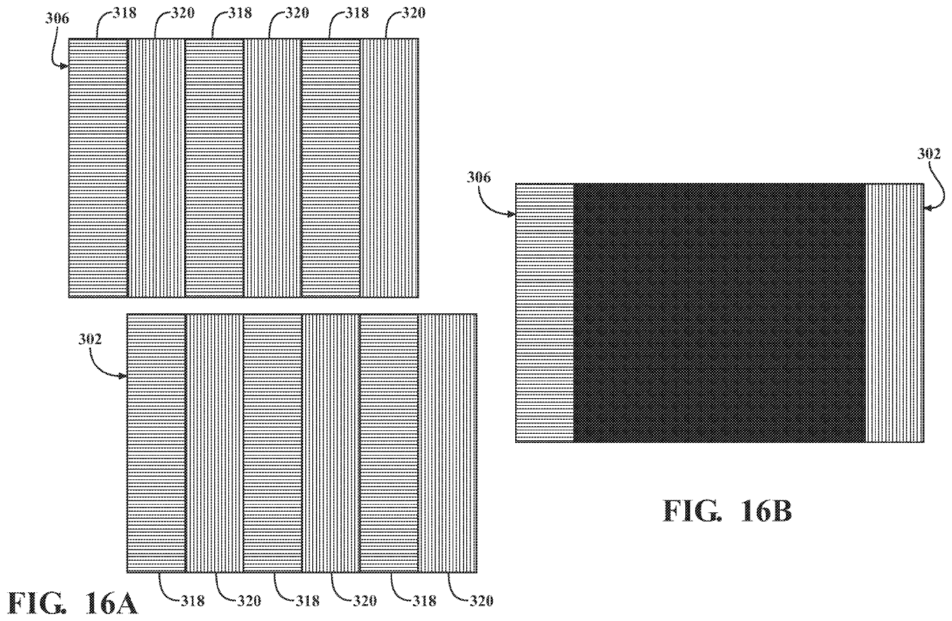

Referring to FIGS. 15A-16B, the first segments 318 may have front and rear surfaces that define polarizing slits between that are arranged in a horizontal orientation. The second segments 320 may have front and rear surfaces that define polarizing slits between the front and rear surfaces. In contrast to the first segments 318, the polarizing slits in the second segments 320 may be arranged in a vertical orientation. Each of the covers 302, 306 include alternating first and second segments 318, 320 arranged in series such that each of the covers 302, 306 include multiple first and second segments 318, 320. When the segments 318, 320 are arranged in series, the segments 318, 320 of the covers 302, 306 may be considered to be plate-like or sheet-like. In other words, the segments 318, 320 of the covers 302, 306 may comprise a thin, almost planar footprint. FIGS. 15A and 15B illustrate the relationship between the first and second covers 302, 306 in the first state and correspond with FIG. 13. Specifically, FIG. 15B shows actual relative positions of the first and second covers 302, 306 in the first state as shown in FIG. 13. FIG. 15A does not show the actual position of the first cover 302 relative to the second cover 306, but is intended to illustrate the horizontal position of the first cover 302 relative to the second cover 308 when the cover assembly 300 is in the first state. In a similar manner, FIGS. 18A and 18B illustrate the relationship between the first and second covers 302, 306 in the second state and correspond with FIG. 14. Specifically, FIG. 16B shows actual relative positions of the first and second covers 302, 306 in the second state as shown in FIG. 14. FIG. 16A does not show the actual position of the first cover 302 relative to the second cover 308, but is intended to illustrate the horizontal position of the first cover 302 relative to the second cover 308 when the cover assembly 300 is in the second state.

As shown in FIGS. 15A-16B, the first and second polarizers may include linear polarizers that transmit desired polarization while reflecting the rest. The polarizer slits in the first and second covers 302, 306 may each include many thin openings arranged parallel to each other. In the first state, light that is polarized along these slits is reflected, while light that is polarized perpendicular to these slits is transmitted. In the second state, as the first and second segments 318, 320 are arranged to be ninety degrees out of phase relative to each other, light is polarized in perpendicular directions to each of the polarizers, which results in approximately zero light being transmitted. The first and second polarizers of the first and second covers 302, 306 may include an acrylic substrate. In some configurations, the polarizers may include UV Grade Fused Silica substrate.

The width of the first and second segments 318, 320 may determine the degree to which the actuator 312 and the first cover 302 must move relative to the second cover 306 to operate the cover assembly 300 between the first and second state. By increasing the number of segments 318, 320 and alternating them in series, the degree may be further reduced. Limiting the movement between the first and second covers 302, 306 to move between the first and second state of the cover assembly 300 mitigates the space required within the housing 38 to accommodate the cover assembly 300 in the first and second states. Furthermore, limiting the movement between the first and second covers 302, 306 to move between the first and second state of the cover assembly 300 reduces the effort required on behalf of the user to operate the cover assembly 300 between the first and second states.

While the polarizing slits in the first and second segments 318, 320 are described above as being arranged in horizontal and vertical orientations, it is contemplated that the polarizing slits may be arranged in other orientations. In order to mitigate light transmissivity in the second state, the polarizing slits in the first segment 318 may be arranged to be ninety degrees out of phase with the polarizing slits in the second segment 320 to best promote a cross polarizing effect approximating zero transmission of light.

A fifth implementation of the cover assembly 400 is shown in FIG. 17. The first and second segments 418, 420 of the covers of the cover assembly 400 may be formed as chevrons in shape. It is contemplated that the first and second segments 418, 420 of the covers of the cover assembly 400 may include other shapes. In some configurations, the polarizers of the covers 302, 306 of the fourth and fifth implementation may be incorporated with the covers 102, 106, 202, 206 of the first and second implementations such that the first and second polarizers of the covers 302, 306 move at the same time to further reduce the degree of actuation necessary to obscure the view through the window.

As discussed above, the transfer valve 74 may be opened to permit waste material to be transferred to the lower waste container 40b from the upper waste container 40a. The dumping protocol may include the waste material being gravity-fed from the upper waste container 40a, through the transfer valve 74, and into the lower waste container 40b. In some cases, the waste material may partially or fully clog the path or volume near or within the transfer valve 74, resulting in a clogged condition to the system 30. For example, a clog may result from blood clots and other buildup of waste material within or near an orifice of the transfer valve 74, which is of relatively smaller diameter than adjacent portions of the upper waste container 40a from which the waste material is being dumped. In the clogged condition, the rate at which waste material is transferred to the lower waste container 40b from the upper waste container 40a may be undesirable, and in some instances, approximate zero (i.e., no waste material leaves the upper waste container 40a). This could lead to delay in resuming the surgical procedure, and/or downtime of the medical waste collection system 30 for service.

The medical waste collection system 30 advantageously provides for alleviating or eliminating the clogged condition by creating a pressure differential between the upper waste container 40a and the lower waste container 40b. This is facilitated with controlling the suction drawn on the waste containers 40a, 40b from the vacuum source 42, and/or with a pump 50. FIG. 18 shows a first or upper vacuum regulator 48a associated with the upper waste container 40a, and a second or lower vacuum regulator 48b associated with the lower waste container 40b such that independent suction may be provided to the upper and lower waste containers 40a, 40b. In other words, the vacuum regulators 48a, 48b may be configured to independently regulate a level of the suction drawn by the vacuum pump 46 on the waste containers 40a, 40b. To unclog, dislodge or otherwise aid the waste contents to be moved through the transfer valve 74 in the clogged condition, the pressure differential may be generated by system control with the controller 86 in manners to be described. Additionally or alternatively, the pressure differential may be generated or assisted by the pump 50 that is separate from the vacuum pump 46.

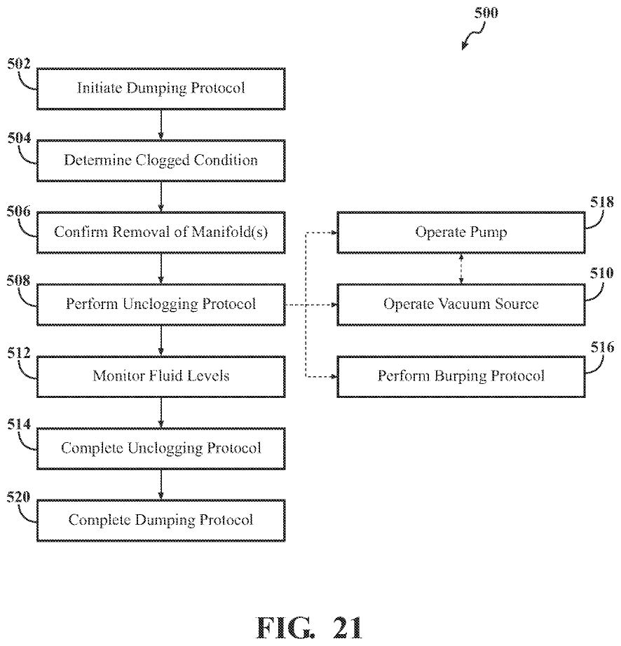

An exemplary method 500 is represented in FIG. 21. The method includes the step of initiating the dumping protocol (step 502). The controller 86 is configured to initiate the dumping protocol based on an input from a user to the control panel 88. For example, the control panel 88 may display information or provide an alert that the fluid volume in the upper waste container 40a exceeds a threshold volume. The threshold volume may be a fixed value, or a determined value based on available volume in view of anticipated fluid collection for one or more upcoming procedures. In another implementation, the controller 86 may initiate the dumping protocol automatically based on certain system conditions, for example, the system 30 not being in use or idle for a predetermined period of time after the threshold volume has been exceeded.

The dumping protocol may include termination operation of the vacuum source 48, and moving the transfer valve 74 to the open position. The waste material, under in the influence of gravity, may start passing through the transfer valve 74 and into the lower waste container 40b. The user may visually observe the dumping through the windows of the waste containers 40a, 40b. It is contemplated that the fluid-level related lighting aspects previously disclosed herein may be used in combination with the dumping protocol to provide further feedback to the progress of the dumping protocol. In particular, the light sources 64 of the light source assemblies 62 associated with each of the upper and lower waste containers 40a, 40b may be selectively deactivated or activated as the fluid volume decreases in the upper waste container 40a, and increases in the lower waste container 40b. For example, the light sources 64 associated with the upper waste container 40a may sequentially deactivate from top to bottom, and the light sources 64 associated with the lower waste container 40b may sequentially activate from bottom to top.

The method includes determining whether the clogged condition exists (step 504). The medical waste collection system 30 includes the level sensor 56a, 56b coupled to each of the upper and lower waste containers 40a, 40b, respectively. In implementation in which there is a single waste container 40, a single level sensor 56 may be used. In another variant, the determinations made by the controller 86 may be based only on the level sensor 56a of the upper waste container 40a despite the presence of the second level sensor 56b. The level sensors 56a, 56b may be coupled to the waste containers 40a, 40b and in electronic communication with the controller 86. The level sensors 56a, 56b are configured to generate a signal indicative of a fluid level of the waste material disposed within the corresponding waste container 40a, 40b. The level sensors 56a, 56b may be similar to those described above or as described in aforementioned International Patent Publication No. WO 2007/070570. It is also contemplated that the level sensors 56a, 56b may include a different design that is capable of generating signals to indicate the fluid level. The controller 86 may be configured to determine a fluid volume of the respective waste containers 40a, 40b based on the corresponding level signals, and a known geometric profile of the waste containers 40a, 40b.

During the dumping protocol in which the transfer valve 74 is in the open position, the controller 86 is configured to determine a rate of change of fluid level (and/or fluid volume) based on level signals received from at least one of the level sensors 56a, 56b. For example, the controller 86 may determine the fluid levels within the upper waste container 40a based on the level signals over a known period of time, and determine the rate as an actual or average change per unit time. The controller 86 may compare the rate of change to a preset fluid transfer rate. The preset fluid transfer rate may be calibration data entered during manufacturing (or through a software update), or entered by a user or service technician on the control panel 88. Alternatively, the controller 86 may determine that the change in fluid level from a first time to a second time does not exceed a preset transfer threshold. The preset transfer threshold may be calibration data entered during manufacturing (or through a software update), or entered by a user or service technician on the control panel 88. In some instances, the rate of change of the fluid level may be broadly understood to implicate a change in fluid level over a period of time.

The controller 86 is repeatedly (e.g., constantly) performing the comparison during the dumping protocol. If the rate of change is above the preset fluid transfer rate, the controller 86 determines that no clogged condition is present, and the controller 86 does not initiate the unclogging protocol. If the rate of change is below the preset fluid transfer rate, the controller 86 determines that the clogged condition may be present. Likewise, if the change in fluid level is above the preset transfer threshold, the controller 86 determines that no clogged condition is present. If the change in fluid level is below the preset transfer threshold, the controller 86 determines the clogged condition may be present.

In addition to the preset fluid transfer level and/or the preset transfer threshold, the controller 86 determines whether the fluid level is below a preset low fluid level. In other words, in the dumping protocol in which there is no clogging, the fluid level rate of change will reduce to zero once the canister is actually empty. Therefore, it is necessary to ensure the low rate of change is merely to the waste container 40a, 40b being empty. If the rate of change of the fluid level is below the preset fluid transfer rate, and the fluid level is above the preset low fluid level, the controller 86 determines the existing of the clogged condition.

The controller 86 may execute a timer or otherwise require the rate of change be below the preset fluid transfer rate for a sufficient period of time before initiating the unclogging protocol. In other words, the controller 86 may be configured to ignore transient, momentary instances where the rate of change is below the preset fluid transfer rate for smooth system control. For example, a blood clot may momentarily obstruct passage of the waste material through the transfer valve 74, but perhaps thereafter cleared from the hydrostatic pressure from the waste material above.

Prior to operating the vacuum source 42 in furtherance of the unclogging protocol, the controller 86 may determine whether a manifold 55 is present in one or both of the manifold receivers 52a, 52b, and in particular, whether a manifold 55 is present in the lower manifold receiver 52b (step 506). As generally appreciated from the fluid circuit schematically represented in FIG. 18, the presence of a manifold in the lower manifold receiver 52b creates a suction path from the vacuum source 42 to the external environment (e.g., the suction tube) through the lower manifold receiver 52b, and the lower waste container 40b. Owing to the principles of fluid dynamics, namely the principle of the path of least resistance, suction on the clogged transfer valve 74 may be insufficient if less resistance is faced from the aforementioned suction path. Removal of the manifold(s) 55 from the manifold receivers 52a, 52b interrupts or seals off the suction path from the waste containers 40a, 40b and the respective manifold receiver 52a, 52b. Details regarding the interruption of the suction path(s) are disclosed in commonly-owned U.S. Pat. No. 7,615,037, issued Nov. 10, 2009, International Patent Publication No. WO 2020/209898, published Oct. 15, 2020, and International Patent Publication No. WO 2022/103832, published May 19, 2022, the entire contents of each being hereby incorporated by reference. With the suction path interrupted between the lower manifold receiver 52b and the lower waste container 40b, suction from the vacuum source 42 on the lower waste container 40b during the unclogging operation creates a more efficient pressure differential, thereby focusing the forces from the pressure differential on the clogged material within the transfer valve 74. Moreover, ensuring that a manifold 55 is not coupled to one of the manifold receivers 52a, 52b prevents the vacuum source 42 from drawing suction at the patient site through the suction lines 80.

Each of the manifold receivers 52a, 52b may include a manifold sensor 54a, 54b coupled to the respective manifold receiver 52a, 52b. The manifold sensors 54a, 54b arc schematically represented in FIG. 18, and are in electronic communication with the controller 86. The manifold sensors 54a, 54b may be configured to generate a manifold signal responsive to the presence of a manifold 55 being received within the respective manifold receiver 52a, 52b. The controller 86 may receive the manifold signal and determine whether a manifold 55 is removably inserted into one of the manifold receivers 52a, 52b based on the manifold signal.

The method 500 includes performing the unclogging protocol (step 508). The controller 86 is configured to permit execution of the unclogging protocol to be described based on the determination that a manifold 55 is not removably inserted into one of the manifold receivers 52a, 52b. If a manifold 55 is determined to be coupled to one of the manifold receivers 52a, 52b and it is attempted to initiate the unclogging protocol, either automatically or through user input, an alert may be generated on the control panel 88. The alert may instruct the user to remove the manifold 55 from the associated manifold receiver 52a, 52b. The alert may include textual or graphical indicia. Once the user removes the manifold 55, the associated manifold sensor 54a, 54b may generate an updated manifold signal indicating the manifold 55 has been removed. The controller 86 may receive the updated manifold signal and the user or the controller 86 may then proceed with the unclogging protocol.

In one implementation, the unclogging protocol includes the controller 86 operating the vacuum source 42 to draw suction on the lower waste container 40b with the transfer valve 74 in the open position (step 510). In some implementations, the controller 86 may operate the vacuum source 42 at a vacuum level of less than 100 mmHg, and more particularly less than 55 mmHg. In other implementations, the controller 86 may operate the vacuum source 42 through a full range of vacuum levels. The negative pressure buildup in the lower waste container 40b creates the aforementioned pressure differential relative to the upper waste container 40a. The pressure differential imparts forces on the waste material that is clogging the transfer valve 74. The forces increase the likelihood that the waste material is drawn from the upper waste container 40a into the lower waste container 40b through the transfer valve 74, thus eliminating the clogged condition.

The controller 86 may be configured to actuate the upper vacuum regulator 48a to terminate suction being drawn on the upper waste container 40a prior to suction being drawn on the lower waste container 40b. The controller 86 may be further configured to actuate the upper vacuum regulator 48a to vent the upper waste container 40a to atmospheric conditions while the vacuum source 42 draws suction on the lower waste container 40b. In this manner, the pressure differential may be better facilitated between the upper and lower waste containers 40a, 40b.

The controller 86 may monitor the fluid level signals as the suction is being drawn on the lower waste container 40b by the vacuum source 42 (step 512). Based on the monitored rate of change, the system 30 may respond dynamically. For example, if the rate of change of the fluid level does not increase to be above the preset fluid transfer rate within a set period of time, the controller 86 may operate the vacuum source 42 to increase the suction on the lower waste container 40b. In other words, the system 30 may create a greater pressure differential if it is determined the waste material has not been unclogged. By contrast, if the rate of change of the fluid level increases to be above the preset fluid transfer rate, the controller 86 may operate the vacuum source 42 to terminate the suction on the lower waste container 40b. Such an increase in the rate of change may indicate that the waste material has been unclogged. The controller 86 may also terminate suction based on the fluid level within the upper waste container 40a decreasing to be below the preset low fluid level. In this instance, it may be necessary to terminate operation of the vacuum source 42 if most or all of the waste material has been dumped into the lower waste container 40b. In such an instance, the rate of change of the fluid level may be below the fluid transfer rate due to a lack of waste material in the upper waste container 40a rather than a clogged condition in the upper waste container 40a.

In another implementation, the system 30 may respond dynamically based on the monitored fluid levels. For example, if the rate and/or change in fluid level is still indicative of a clogged condition, the controller 86 may operate the vacuum source 42 at different levels based on the fluid level relative to one or more preset fluid levels. In one implementation, the controller may operate the vacuum source 42 at a higher vacuum level when the fluid level is determined to be nearly full and at a lower vacuum level when the fluid level is determined to be near empty regardless of the change in fluid level over time.

In another implementation, controller 86 may monitor the fluid level signals as the suction is being drawn on the lower waste container 40b by the vacuum source 42 and the controller may assess the change in fluid level in a rolling fashion. For example, the controller 86 may determine a change in fluid level from a first time to a second time, from the second time to a third time, from the third time to a fourth time, and so on. The controller 86 may determine the change in fluid level between the first and second times is sufficient enough not to trigger a clogged condition. The controller evaluates the change in fluid level from the second time to the third time and may determine the change in fluid level is indicative of a clogged condition. In some instances, the controller 86 may use different prese transfer thresholds for different period of times to determine the clogging condition exists. In some instances, the time periods are equal durations. In other instances, the time periods are not equal. In some instances, the time periods change based on whether the controller determined the existence of the clogged condition in the preceding time period. The controller 86 may shorten the time periods when a clogged condition is determined so that operation of vacuum source 42 to remove the clogged condition is limited to as little time as possible.

The method 500 may include completing the unclogging protocol (step 514). The controller 86 may also terminate operation of the vacuum source 42 based on the rate of change of the fluid level not increasing to be above the preset fluid transfer rate within a set period of time. In this instance, operation of the vacuum source 42 may not be sufficient to unclog the upper waste container 40a. The controller 86 may be configured to provide an error condition alert on the control panel 88 to indicate to the user that the upper waste container 40a remains clogged. Additionally or alternatively, the error condition may be transmitted to a service technician or another entity through a network connection. The controller 86 may prevent further operation of the medical waste collection system 30 until the error condition is satisfactorily addressed.

The pressure differential generated with the unclogging protocol discussed above may generally provide constant forces on the waste material, the magnitude of which may be based on the level of the suction from the vacuum source 42. In one variant, a burping protocol may be performed (step 516) in which the suction on the lower waste container 40b is alternatingly started and stopped, increased and decreased, or combinations thereof. In other words, the controller 86 may be configured to operate the vacuum source 42 and/or the lower vacuum regulator 48b at fixed or varied intervals and/or fixed or varied vacuum levels in order to promote agitation within the upper waste container 40a. The transient spikes in forces on the clogged material may be more likely to dislodge the clog from within the transfer valve 74. The burping protocol is an optional step.

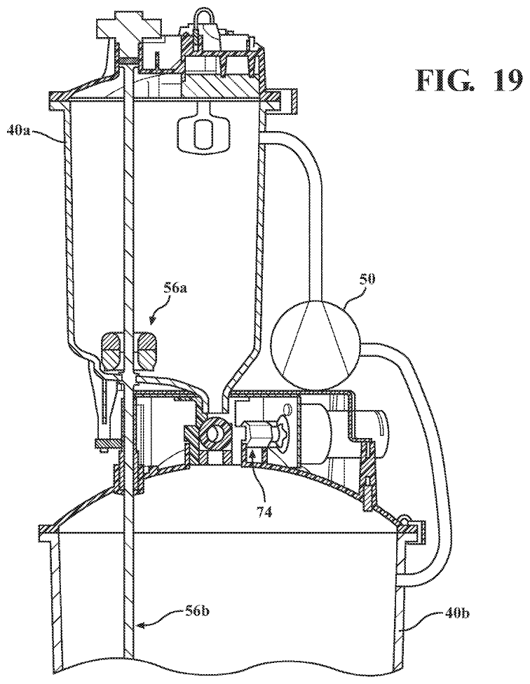

In another embodiment, the medical waste collection system 30 may include the aforementioned pump 50, FIGS. 18 and 19 show the pump 50 is disposed on the base 32 and separate from the vacuum pump 46. The pump 50 is in fluid communication with at least the upper waste container 40a, and in electronic communication with the controller 86. The pump 50 may be a positive pressure pump operable to force air into the upper waste container 40a to create or increase the pressure differential (step 518). In one variant, the pump 50 also is in fluid communication with the lower waste container 40b, and operable to decrease the pressure in the lower waste container 40b to create the pressure differential. For example, the pump 50 may draw air from the lower waste container 40b and urge it into the upper waste container 40a. Alternatively, multiple pumps may be provided.

The pump 50 may be incorporated into the unclogging protocol similar to that previously described. For example, the controller 86 may prevent operation of the pump 50 based on the determination that a manifold 55 is removably inserted into one of the manifold receivers 52a, 52b. In this instance, ensuring that a manifold 55 is not coupled to one of the manifold receivers 52a, 52b prevents an occurrence of the pump 50 forcing air to the patient site through the suction lines 80. For another example, the controller 86 is configured to operate the pump 50 to direct air into the upper waste container 40a based on the determination that the rate of change of the fluid level is less than a preset fluid transfer rate, and that the fluid level is greater than a preset low fluid level.

In an exemplary method, the controller 86 actuates the transfer valve 74 to an open position to permit waste material to be transferred from the upper waste container 40a to the lower waste container 40b. The controller 86 receives fluid level signals from at least one of the level sensors 56a, 56b. Based on the level signals, the controller 86 determines a fluid level within the upper waste container 40a and a rate of change of fluid level within at least one of the upper waste container 40a and the lower waste container 40b. If the rate of change of fluid level is less than a preset fluid transfer rate and if the fluid level is greater than a preset low fluid level, the controller 86 determines the presence of the clogged condition. The controller 86 may actuate the upper vacuum regulator 48a to terminate suction being drawn on the upper waste container 40a prior to the step of operating the pump 50. The controller 86 operates the pump 50 to force air into the upper waste container 40a. The pressure differential is increased to unclog and force the waste material through the transfer valve 74.

The controller 86 may be configured to monitor the fluid level signals as the pump 50 is being operated. In some instances, the controller 86 decreases or increases the speed of the pump 50 (i.e., the amount of air being forced into the upper waste container 40a) based on the rate of change of the fluid level not increasing or increases, respectively, to above the preset fluid transfer rate within a set period of time. The controller 86 may also terminate operation of the pump 50 based on the fluid level within the upper waste container 40a decreasing to below the preset low fluid level, indicative that most or all the waste material has been dumped into the lower waste container 40b. In instances in which operation of the pump 50 is insufficient to unclog the upper waste container 40a, the controller 86 may terminate operation of the pump 50 and provide the error condition alert on the control panel 88.

In some implementations, control of the pump 50 may be used in combination with the system control of the vacuum source 42 in the manners previously described. In particular, the controller 86 may be configured to simultaneously operate the vacuum pump 46, the lower vacuum regulator 48b, and/or the pump 50 in a coordinated manner to generate the desired or increased pressure differential. The coordinated operation is particularly advantageous in instances where the pump 50 is not coupled to and in fluid communication with the lower waste container 40b to prevent a condition where the upper and lower waste containers 40a, 40b are under pressure. The coordinated operation may also be implemented with the burping protocol to alternatingly start and stop, and/or increase and decrease the positive pressure being provided to the upper waste container 40a and the negative pressure being provided to the lower waste container 40. Preferably, with successful unclogging of the transfer valve 74, the dumping protocol may then be completed (step 520).

Several configurations have been discussed in the foregoing description. However, the configurations discussed herein are not intended to be exhaustive or limit the disclosure to any particular form. The terminology which has been used is intended to be in the nature of words of description rather than of limitation. Many modifications and variations are possible in light of the above teachings and may be practiced otherwise than as specifically described.

1. A medical waste collection system for collecting waste material, the medical waste collection system comprising:
a waste container;
a vacuum source configured to provide a vacuum on the waste container;
a chassis supporting the waste container and the vacuum source;
a level sensor coupled to the waste container and configured to generate a signal indicative of a fluid level of the waste material disposed within the waste container;
a light source assembly comprising a plurality of light sources, each of the plurality of light sources configured to be selectively operable to illuminate an interior of the waste container; and
a controller coupled to the level sensor and the light source assembly, the controller configured to operate the plurality of light sources based on the signal such that a portion of the interior of the waste container disposed beneath the fluid level of the waste material is illuminated.

2. The medical waste collection system of claim 1, wherein the controller is further configured to operate the light source assembly to illuminate the waste container with only one or more of the plurality of light sources disposed beneath the fluid level of the waste material in the waste container based on the signal.

3. The medical waste collection system of claim 1, wherein the controller comprises memory storing data associating a vertical position of each of the plurality of light sources to a corresponding predefined fluid level.

4. The medical waste collection system of claim 3, wherein the controller is configured to:
receive the signal from the level sensor;
determine the fluid level of waste material disposed within the waste container based on the signal;
compare the data accessed from the memory with the determined fluid level; and
operate the plurality of light sources based on the comparison.

5. The medical waste collection system of claim 1, wherein plurality of light sources are spaced apart from one another vertically with respect to gravity; and, optionally, the spacing being equidistant.

6. The medical waste collection system of claim 1, wherein the light source assembly is a light strip comprising a substrate with a plurality of light sources being light emitting diodes (LEDs).

7. The medical waste collection system of claim 6, wherein the chassis comprises a housing defining a viewing window through which the waste container is visible, and wherein the light strip is disposed between the chassis and the waste container adjacent to a side of the viewing window.

8. The medical waste collection system of claim 7, wherein the light assembly further comprises a second light strip disposed between the housing and the waste container adjacent to an opposing side of the viewing window.

9. The medical waste collection system of claim 1, wherein the plurality of light sources are configured to emit light in a variable light intensity, wherein the memory stores the data further associating the predefined fluid levels with predefined light intensities, and wherein the controller is further configured to operate the plurality of light sources at the predefined light intensities based on the signal such that light sources disposed above the fluid level are operated at a first intensity and light sources disposed beneath the fluid level are operated at a second intensity greater than the first intensity.

10. The medical waste collection system of claim 1, wherein the plurality of light sources are configured to emit light in a variable light color, wherein the memory stores the data further associating the predefined fluid levels with predefined light colors, and wherein the controller is further configured to operate the plurality of light sources at one or more of the predefined light colors based on the signal.

11. The medical waste collection system of claim 1, further comprising a user interface in communication with the controller and configured to receive a full illumination input such that the controller operates all of the plurality of light sources regardless of the fluid level.

12. The medical waste collection system of claim 1, wherein the plurality of light sources are configured to emit light in at least one of a variable light intensity and a variable light color, the system further comprising a user interface in communication with the controller and configured to receive a light characteristic input such that the controller operates the plurality of light sources at a selected color or a selected intensity.

13-103. (canceled)

104. A medical waste collection system for collecting waste material, the medical waste collection system comprising:
a waste container;
a vacuum source configured to provide a vacuum on the waste container;
a chassis supporting the waste container and the vacuum source;
a level sensor coupled to the waste container and configured to generate a signal indicative of a fluid level of the waste material disposed within the waste container;
a light source assembly comprising a plurality of light sources, each of the plurality of light sources configured to be selectively operable to illuminate an interior of the waste container; and
a controller coupled to the level sensor and the light source assembly, the controller configured to:
receive the signal from the level sensor;
determine the fluid level of waste material disposed within the waste container based on the signal;
compare the data accessed from memory with the determined fluid level; and
operate the plurality of light sources based on the comparison.

105. The medical waste collection system of claim 104, wherein the plurality of light sources are configured to emit light in a variable light intensity, wherein the memory stores the data further associating the predefined fluid levels with predefined light intensities, and wherein the controller is further configured to operate the plurality of light sources at the predefined light intensities based on the signal such that light sources disposed above the fluid level are operated at a first intensity and light sources disposed beneath the fluid level are operated at a second intensity greater than the first intensity.

106. The medical waste collection system of claim 104, wherein the plurality of light sources are configured to emit light in a variable light color, wherein the memory stores the data further associating the predefined fluid levels with predefined light colors, and wherein the controller is further configured to operate the plurality of light sources at one or more of the predefined light colors based on the signal.

107. A medical waste collection system for collecting waste material, the medical waste collection system comprising:
a waste container;
a vacuum source configured to provide a vacuum on the waste container;
a chassis supporting the waste container and the vacuum source;
a level sensor coupled to the waste container and configured to generate a signal indicative of a fluid level of the waste material disposed within the waste container;
a light source assembly comprising a plurality of light sources, each of the plurality of light sources configured to be selectively operable to illuminate an interior of the waste container; and
a controller coupled to the level sensor and the light source assembly, the controller comprises memory storing data associating a vertical position of each of the plurality of light sources to a corresponding predefined fluid level.

108. The medical waste collection system of claim 107, wherein plurality of light sources are spaced apart from one another vertically with respect to gravity; and, optionally, the spacing being equidistant.

109. The medical waste collection system of claim 107, wherein the light source assembly is a light strip comprising a substrate with a plurality of light sources being light emitting diodes (LEDs).

110. The medical waste collection system of claim 109, wherein the chassis comprises a housing defining a viewing window through which the waste container is visible, and wherein the light strip is disposed between the chassis and the waste container adjacent to a side of the viewing window.
